SEQUENCE LISTING (8 sequences)

SEQ ID NO: 1 - DNA, 46 bp, Artificial Sequence
  Synthetic Construct
cacatgcgtctatgtagtggagccttagagagtagtacttccgact

SEQ ID NO: 2 - DNA, 49 bp, Artificial Sequence
  Synthetic Construct
cacatgcgtctatatagtggagccttgtagtacagcagcagcattgagg

SEQ ID NO: 3 - DNA, 47 bp, Artificial Sequence
  Synthetic Construct
cacatgcgtctatttagtggagccttcaatctagtatcagtggcgca

SEQ ID NO: 4 - DNA, 46 bp, Artificial Sequence
  Synthetic Construct
cacatgcgtctatctagtggagccttgggccttattccggtgctat

SEQ ID NO: 5 - DNA, 20 bp, Artificial Sequence
  Synthetic Construct
  misc_feature (1)..(1): Cy3
agtcggaagtactactctct

SEQ ID NO: 6 - DNA, 23 bp, Artificial Sequence
  Synthetic Construct
  misc_feature (1)..(1): Cy5
cctcaatgctgctgctgtactac

SEQ ID NO: 7 - DNA, 21 bp, Artificial Sequence
  Synthetic Construct
  misc_feature (1)..(1): AF488
tgcgccactgatactagattg

SEQ ID NO: 8 - DNA, 20 bp, Artificial Sequence
  Synthetic Construct
  misc_feature (1)..(1): TexR
atagcaccggaataaggccc

(19) United States
(12) Patent Application Publication
(10) Pub. No.: US 2022/0195498 A1
(43) Pub. Date: Jun. 23, 2022

(54) METHODS AND COMPOSITIONS FOR ANALYTE DETECTION

(71) Applicant: 10x Genomics, Inc., Pleasanton, CA (US)

(72) Inventors: Malte KÜHNEMUND, Stockholm (SE); Xiaoyan QIAN, Huddinge (SE)

(21) Appl. No.: 17/559,511

(22) Filed: Dec. 22, 2021

Related U.S. Application Data
(60) Provisional application No. 63/130,276, filed on Dec. 23, 2020.

(51) Int. Cl. C12Q 1/6818

(52) U.S. Cl. CPC C12Q 1/6818

(57) ABSTRACT

In some aspects, the present disclosure relates to methods for reducing the crowding of signals, for example optical crowding, that can occur when nucleic acids are detected in a sample in multiplex, which can make it difficult to resolve individual signals and can lead to a reduced dynamic range. In some aspects, the present disclosure relates to methods for reducing signal crowding in the detection of multiple target nucleic acid sequences in a sample, e.g., using hybridization probes, wherein signal crowding from said hybridization probes is reduced. The methods herein have particular applicability in the detection of barcode sequences by sequencing-by-hybridization (SBH) methods, including those relying on combinatorial labelling schemes and decoding of the barcodes by sequential cycles of decoding using hybridization probes. Also provided are kits comprising probes for use in such methods.

### CROSS-REFERENCE TO RELATED APPLICATIONS

This application claims priority to U.S. Provisional Patent Application No. 63/130,276, filed Dec. 23, 2020, entitled “METHODS AND COMPOSITIONS FOR ANALYTE DETECTION,” which is herein incorporated by reference in its entirety for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 202412006100SEQLIST.TXT, date recorded: Dec. 20, 2021, size: 1,951 bytes).

### FIELD

The present disclosure generally relates to methods and compositions for detecting a plurality of molecules of one or more analytes in a sample.

### BACKGROUND

In multiplex assays where multiple signals are detected simultaneously, it is important that each individual signal can be distinguished from one another so that as much information as possible can be collected from the assays. For example, in microscopy-based optical assays, individual “spots” emitting optical signals often need to be resolved from adjacent spots in a sample. However, resolving a large number of signals of varying strengths remains challenging, and improved methods are needed. The present disclosure addresses these and other needs.

### SUMMARY

In some embodiments, provided herein is a method for nucleic acid sequence detection, comprising: (a) in any suitable order, contacting a sample, a first probe capable of hybridizing to a first target nucleic acid sequence, a second probe capable of hybridizing to a second target nucleic acid sequence, and an interfering agent, wherein: the first and second target nucleic acid sequences are different, and hybridization of the first probe to the first target nucleic acid sequence is not interfered by the interfering agent, whereas hybridization of the second probe to the second target nucleic acid sequence is interfered by the interfering agent; and (b) detecting a signal indicative of the hybridization of the first probe to the first target nucleic acid sequence in the sample, whereas a signal indicative of the hybridization of the second probe to the second target nucleic acid sequence in the sample is not detected or is detected at a lower level compared to a reference signal detected without the interfering agent interfering with hybridization, thereby detecting the first target nucleic acid sequence in the sample. In some embodiments, the first and/or second probes are contacted with the interfering agent before contacting the sample.

In any of the preceding embodiments, the sample can comprise a plurality of first target nucleic acid sequences that are different from each other, and the contacting step can comprise contacting the sample with a plurality of first probes each capable of hybridizing to one of the plurality of first target nucleic acid sequences.

In any of the preceding embodiments, the sample can comprise a plurality of second target nucleic acid sequences that are different from each other and different from the first target nucleic acid sequence(s), and the contacting step can comprise contacting the sample with a plurality of second probes each capable of hybridizing to one of the plurality of second target nucleic acid sequences.

In any of the preceding embodiments, the interfering agent can interfere with hybridization of two or more second probes to the corresponding second target nucleic acid sequences.

In any of the preceding embodiments, the first and second probes can be contacted with the interfering agent to form a second probe/interfering agent hybridization complex or a second target nucleic acid sequence/interfering agent hybridization complex, before the sample is contacted with the first and second probes and the interfering agent, optionally wherein the contacting step comprises contacting the sample with a plurality of interfering agents that are the same or different.

In any of the preceding embodiments, the plurality of interfering agents can comprise interfering agents that interfere with hybridization of the same second probe to the corresponding second target nucleic acid sequence.

In any of the preceding embodiments, the plurality of interfering agents can comprise interfering agents that each interferes with hybridization of a different second probe to the corresponding second target nucleic acid sequence, optionally wherein the different second probes share a binding sequence that hybridizes to the second target nucleic acid sequence but comprise different binding sequences for different detectably labelled detection oligonucleotides.

In any of the preceding embodiments, the plurality of interfering agents can comprise an interfering agent that interferes with hybridization of two or more second probes to the corresponding second target nucleic acid sequences.

In any of the preceding embodiments, interfering agent can hybridize to the second probe but not to the first probe.

In any of the preceding embodiments, the interfering agent can prevent the second probe from hybridizing to the second target nucleic acid sequence.

In any of the preceding embodiments, the interfering agent can displace the second probe which is hybridized to the second target nucleic acid sequence.

In any of the preceding embodiments, the interfering agent can hybridize to the second target nucleic acid sequence but not to the first target nucleic acid sequence.

In any of the preceding embodiments, the interfering agent can prevent the second probe from hybridizing to the second target nucleic acid sequence, optionally wherein the interfering agent comprises a sequence complementary to a sequence of the second probe, or a sequence complementary to a sequence of the second target nucleic acid sequence.

In any of the preceding embodiments, the interfering agent can displace the second probe which is hybridized to the second target nucleic acid sequence.

In any of the preceding embodiments, the first probe and/or the second probe can be detectably labelled.

In any of the preceding embodiments, the first probe and/or the second probe can be covalently or non-covalently coupled to a fluorescent label.

In any of the preceding embodiments, the first probe and/or the second probe can directly or indirectly bind to a detectably labelled detection probe.

In any of the preceding embodiments, the first probe and/or the second probe can comprise one or more overhangs that do not hybridize to the first and second target nucleic acid sequences, respectively.

In any of the preceding embodiments, at least one of the one or more overhangs can be capable of hybridizing to a detectably labelled detection probe.

In any of the preceding embodiments, the second probe can comprise an overhang that is capable of hybridizing to a sequence of the interfering agent. In some embodiments, hybridization of the interfering agent to the overhang of the second probe can initiate a strand displacement reaction whereby the interfering agent hybridizes to the second probe and displaces it from the second target nucleic acid sequence.

In any of the preceding embodiments, the first and second target nucleic acid sequences can correspond to a first analyte and a second analyte, respectively, in the sample.

In any of the preceding embodiments, the first and/or second analytes can be DNA (e.g., genomic DNA or cDNA), RNA (e.g., mRNA), or protein.

In any of the preceding embodiments, the first analyte can be less abundant than the second analyte in the sample.

In any of the preceding embodiments, the number of molecules comprising the second analyte can be at least 2, 5, 10, 20, 50, 100, 200, 500, or 1,000 times of the number of molecules comprising the first analyte.

In any of the preceding embodiments, the signals detected in the detecting step (step (b)) can be optical (e.g., fluorescent) signals, and the signal indicative of the hybridization of the first probe to the first target nucleic acid sequence may not overlap (e.g., may not spatially overlap) with the signal indicative of the hybridization of the second probe to the second target nucleic acid, if detected.

In any of the preceding embodiments, the signal indicative of the hybridization of the first probe to the first target nucleic acid sequence can overlap (e.g., can spatially overlap) with the reference signal, wherein the reference signal is a signal indicative of hybridization of the second probe to the second target nucleic acid sequence in the absence of the interfering agent.

In any of the preceding embodiments, the first and/or second analytes can be selected prior to the contacting step.

In any of the preceding embodiments, the method can further comprise selecting the first and second probes corresponding to the first and second analytes, respectively.

In any of the preceding embodiments, the method can further comprise selecting the interfering agent which hybridizes to the second probe but does not hybridize to the first probe.

In any of the preceding embodiments, the method can further comprise selecting the interfering agent which hybridizes to the second target nucleic acid sequence but does not hybridize to the first target nucleic acid sequence.

In any of the preceding embodiments, the method can further comprise removing the first probe hybridized to the first target nucleic acid sequence in the sample.

In any of the preceding embodiments, the method can further comprise contacting the sample with the second probe but not the interfering agent, and detecting a signal indicative of the hybridization of the second probe to the second target nucleic acid sequence in the sample, thereby detecting the second target nucleic acid sequence in the sample.

In any of the preceding embodiments, the method can further comprise prior to the contacting step, contacting the sample with the first and second probes but not with the interfering agent, wherein a signal indicative of the hybridization of the first probe to the first target nucleic acid sequence in the sample is crowded out by the reference signal which is indicative of the hybridization of the second probe to the second target nucleic acid sequence in the sample without the interfering agent.

In any of the preceding embodiments, the first and/or second target nucleic acid sequences can be comprised in nucleic acid analytes, e.g., a DNA (e.g., genomic DNA or a cDNA of an mRNA) or RNA analyte in the sample, optionally wherein the first and/or second probes are circular or circularizable (e.g., padlock) probes and the interfering agent does not facilitate circularization of the circularizable probes.

In any of the preceding embodiments, the first and/or second target nucleic acid sequences can be comprised in a product (e.g., a hybridization product, a ligation product, an extension product (e.g., by a DNA or RNA polymerase), a replication product, a transcription/reverse transcription product, and/or an amplification product such as a rolling circle amplification (RCA) product) of a nucleic acid analyte in the sample.

In any of the preceding embodiments, the first and/or second target nucleic acid sequences can be comprised in a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent or a polynucleotide probe that directly or indirectly binds to an analyte in the sample.

In any of the preceding embodiments, the labelling agent can comprise a reporter oligonucleotide comprising one or more barcode sequences, and the amplification product can comprise multiple copies of the one or more barcode sequences.

In any of the preceding embodiments, the first and/or second target nucleic acid sequences can be comprised in an RCA product of a circular or padlock probe that hybridizes to a DNA (e.g., genomic DNA or a cDNA of an mRNA) or RNA analyte in the sample.

In any of the preceding embodiments, the RCA products of a plurality of different mRNA and/or cDNA analytes can be analyzed, a barcode sequence in a particular circular or padlock probe uniquely corresponds to a particular mRNA or cDNA analyte, and the particular circular or padlock probe can further comprise an anchor sequence that is common among circular or padlock probes for a subset of the plurality of different mRNA and/or cDNA analytes.

In any of the preceding embodiments, the labelling agent can directly or indirectly binds to a non-nucleic acid analyte in the sample, e.g., a protein analyte, a carbohydrate analyte, and/or a lipid analyte.

In any of the preceding embodiments, the labelling agent can comprise a reporter oligonucleotide conjugated to an antibody or antigen binding fragment thereof that binds to a protein analyte, and the first and/or second target nucleic acid sequences can be comprised in an RCA product of a circular or padlock probe that hybridizes to the reporter oligonucleotide.

In any of the preceding embodiments, molecules comprising the first and/or second target nucleic acid sequences can be immobilized in the sample.

In any of the preceding embodiments, molecules comprising the first and/or second target nucleic acid sequences can be crosslinked to one or more other molecules in the sample, a matrix such as a hydrogel, and/or one or more functional groups on a substrate.

In any of the preceding embodiments, the first and/or second target nucleic acid sequences can be detected in situ in the sample.

In any of the preceding embodiments, the first and/or second target nucleic acid sequences can comprise a barcode sequence.

In any of the preceding embodiments, the barcode sequence in each first or second target nucleic acid sequence can correspond to a unique signal code sequence.

In any of the preceding embodiments, a set of probes can be provided for decoding of each signal code sequence for each target nucleic acid sequence, each probe in a set comprising the same recognition sequence that hybridizes to the target nucleic acid sequence and a detection hybridization region (also referred to herein as a reporter probe binding site) or the absence of a detection hybridization region, wherein the detection hybridization region or the absence thereof may be the same or different among probes in the set, wherein the detection hybridization region, if present, is specific for a detection probe (also referred to herein as a reporter probe) comprising a detectable label, and wherein the set of probes can be used sequentially in multiple cycles of decoding in a pre-determined sequence which corresponds to the signal code sequence.

In any of the preceding embodiments, a given cycle of decoding can comprise contacting the sample with a probe library comprising a probe of each set of probes, wherein the probe of each set corresponds to the given cycle of decoding.

In any of the preceding embodiments, the method can comprise contacting the sample with an interfering agent or a set of interfering agents in multiple cycles of decoding, wherein the same interfering agent or set of interfering agents is used with the probe library in each cycle of decoding.

In any of the preceding embodiments, the interfering agent or an interfering agent of the set of interfering agents can interfere with hybridization of different second probes to the same corresponding second target nucleic acid sequences in different cycles of decoding.

In any of the preceding embodiments, the same set of interfering agents can be used with the probes in each cycle of decoding.

In any of the preceding embodiments, n sets of probes can be used to decode signal code sequences for target nucleic acid sequences T1, . . . , Tk, . . . , Tn, in m cycles, and Probe Set 1 can comprise P11, . . . , P1j, . . . , and P1m, Probe Set k can comprise Pk1, . . . , Pkj, . . . , and Pkm, Probe Set n can comprise Pn1, . . . , Pnj, . . . , and Pnm, j, k, m, and n are integers, 2≤j≤m, and 2≤k≤n, wherein the sample can be contacted with Probe Library P11, . . . , Pk1, . . . , and Pn1 in Cycle 1, with Probe Library P1j, . . . , Pkj, . . . , and Pnj in Cycle j, and with Probe Library P1m, . . . , Pkm, . . . , and Pnm in Cycle m, wherein each probe can be detectable by a fluorescently labelled reporter probe, and the fluorescent signals for different probes in each probe set or each probe library can be of the same or different colors, and wherein in one or more of Cycle 1 through Cycle m, the sample can be contacted with an interfering agent (e.g., an interfering oligo) that hybridizes to a target nucleic acid sequence or the corresponding probe(s), and the interfering agent (e.g., an interfering oligo) is not fluorescently labelled.

In any of the preceding embodiments, the interfering agent (e.g., an interfering oligo) can hybridize to the target nucleic acid sequence and can prevent, compete with, and/or displace probe(s) from hybridizing to the target nucleic acid sequence, wherein the interfering agent (e.g., an interfering oligo) can be provided at a higher concentration than probe(s) for the target nucleic acid sequence.

In any of the preceding embodiments, the interfering agent (e.g., an interfering oligo) can hybridize to probe(s) for the target nucleic acid sequence, thereby preventing, competing with, and/or displacing the target nucleic acid sequence from hybridizing to the probe(s).

In any of the preceding embodiments, the probe library for one of Cycle 1 through Cycle m, and the interfering agent (e.g., an interfering oligo) if used in that cycle, can be removed prior to contacting the sample with the probe library for the next cycle and optionally another interfering agent.

In any of the preceding embodiments, the interfering agent (e.g., an interfering oligo) and the another interfering agent (e.g., an interfering oligo) can interfere with the hybridization of the same target nucleic acid sequence to the probes for that target nucleic acid sequence.

In any of the preceding embodiments, target nucleic acid sequences T1, . . . , Tk, . . . , Tn can comprise barcode sequences B1, . . . , Bk, . . . , Bn, respectively.

In any of the preceding embodiments, each probe in Probe Set 1, . . . , Probe Set k, . . . , and Probe Set n can comprise recognition sequences R1, . . . , Rk, . . . , Rn, that hybridizes to barcode sequences B1, . . . , Bk, . . . , Bn, respectively.

In any of the preceding embodiments, in each of Cycle 1 through Cycle m, the sample can be contacted with interfering agents (e.g., interfering oligos) that hybridize to one or more of the recognition sequences or one or more of the barcode sequences, whereby fluorescent signals corresponding to the one or more target nucleic acid sequences are not detected or are detected at a lower level compare to without the interfering agents (e.g., interfering oligos) in Cycle 1 through Cycle m.

In any of the preceding embodiments, in each of Cycle 1 through Cycle m, the sample can be contacted with the same interfering agents (e.g., interfering oligos) that hybridize to the recognition sequence(s) corresponding to one or more target nucleic acid sequences.

In any of the preceding embodiments, in each of Cycle 1 through Cycle m, the sample can be contacted with the same interfering agents (e.g., interfering oligos) that hybridize to the barcode sequence(s) of one or more target nucleic acid sequences.

In any of the preceding embodiments, the method can further comprise contacting the sample, in a cycle other than Cycle 1 through Cycle m, with one or more probes that hybridize to the one or more target nucleic acid sequences in the absence of interfering agents (e.g., interfering oligos), wherein the one or more probes can be detected in the sample.

In any of the preceding embodiments, the signal code sequence for each target nucleic acid sequence can comprise signal codes corresponding to the fluorescent signals (or lack thereof) from probes in Cycle 1 through Cycle m.

In any of the preceding embodiments, the lack of fluorescent signals in one or more cycles can be due to the interfering agents (interfering oligos).

In any of the preceding embodiments, the first and second target nucleic acid sequences can be barcodes sequences in rolling circle amplification products.

In some embodiments, provided herein is a method for detecting multiple analytes, comprising: a) determining highly expressed/abundant analytes in a sample; b) select interfering agents to the highly expressed/abundant analytes; c) contacting the sample with interfering agents to the highly expressed/abundant analytes; and d) detecting signals in the sample, wherein signals indicative of the highly expressed/abundant analytes in the sample are not detected or are detected at a lower level compared to a reference signal indicative of the same highly expressed/abundant analytes detected in the absence of the interfering agents.

In any of the preceding embodiments, the selected interfering agents can be interfering probes that hybridize to circularizable (e.g., padlocks) probes that each hybridizes to a DNA or RNA sequence of the highly expressed/abundant analytes, thereby interfering with the ligation of the circularizable (e.g., padlock probes) and generation of rolling circle amplification products indicative of the highly expressed/abundant analytes.

In any of the preceding embodiments, the selected interfering agents can be interfering probes that hybridize to a DNA or RNA sequence of the highly expressed/abundant analytes or a complement thereof, thereby interfering with the hybridization of padlocks probes to the DNA or RNA sequence, ligation of the circularizable (e.g., padlock) probes, and/or generation of rolling circle amplification products indicative of the highly expressed/abundant analytes. In some instances, after lower expressed genes are decoded, the highly expressed/abundant analytes may be detected by providing circularizable probes to the sample (without interfering agents), performing ligation of the probes, and generating rolling circle amplification products indicative of the highly expressed/abundant analytes in order to visualize these the highly expressed/abundant analytes. In some cases, various highly expressed/abundant analytes can be detected individually in a separate decoding round.

In any of the preceding embodiments, the selected interfering agents can be interfering probes that hybridize to hybridization probes that each hybridizes to a rolling circle amplification product indicative of a highly expressed/abundant analyte, thereby preventing the hybridization probes for the highly expressed/abundant analytes from hybridizing to the corresponding rolling circle amplification products in the sample, optionally wherein each hybridization probe comprises (i) a sequence that hybridizes to a barcode sequence in the corresponding rolling circle amplification product and (ii) a non-hybridizing overhang.

In any of the preceding embodiments, the interfering probes can be contacted with the hybridization probes prior to contacting with the sample.

In any of the preceding embodiments, the selected interfering agents can be interfering probes that hybridize to rolling circle amplification products indicative of the highly expressed/abundant analytes, thereby preventing the rolling circle amplification products from hybridizing to the hybridization probes for the highly expressed/abundant analytes in the sample, optionally wherein each hybridization probe comprises (i) a sequence that hybridizes to a barcode sequence in the corresponding rolling circle amplification product.

In any of the preceding embodiments, the selected interfering agents can be interfering probes that hybridize to hybridization probes that each hybridizes to a rolling circle amplification product indicative of a highly expressed/abundant analyte, thereby preventing detection probes from hybridizing to the hybridization probes for the highly expressed/abundant analytes in the sample, wherein the interfering probes do not interfere with hybridization of the hybridization probes to the rolling circle amplification products in the sample.

In some aspects, provided herein is a method for nucleic acid sequence detection, comprising: (a) in any suitable order, contacting a sample, a first probe capable of hybridizing to a first target nucleic acid sequence, a second probe capable of hybridizing to a second target nucleic acid sequence, and an interfering agent, wherein: the first and second target nucleic acid sequences are different, the first probe and the second probe are each associated with a detectable label, which may be the same or different among the first and second probe; the interfering agent is a probe comprising a hybridization region and a quencher moiety; the second probe, but not the first probe, comprises a sequence complementary to the hybridization region of the interfering agent, wherein the interfering agent hybridizes to the sequence of the second probe and quenches the detectable label associated with the second probe; and (b) detecting a signal indicative of the hybridization of the first probe to the first target nucleic acid sequence in the sample, whereas a signal indicative of the hybridization of the second probe to the second target nucleic acid sequence in the sample is inhibited, thereby detecting the first target nucleic acid sequence in the sample.

In some embodiments, the first probe and second probe can each comprise a detection hybridization region, which may be the same or different among the first probe and the second probe, which can be specific for a detection probe comprising the detectable label.

In some embodiments, the sequence complementary to the hybridization region of the interfering agent can correspond to the second target nucleic acid.

In some aspects, provided herein is a kit for nucleic acid sequence detection, the kit comprising: (a) a plurality of hybridization probes comprising different hybridization probes each specific for a different target nucleic acid sequence, wherein each hybridization probe has a recognition sequence complementary to a sequence within its target nucleic acid sequence, and is capable of generating a signal by means of which it can be detected; and (b) an interfering agent comprising a sequence capable of hybridizing to a sequence within a selected hybridization probe of the plurality of hybridization probes, or to a sequence within the target nucleic acid sequence of the selected hybridization probe, wherein hybridization of the interfering agent to the selected hybridization probe or corresponding target nucleic acid sequence interferes with hybridization of the selected hybridization probe and/or generation of the signal by the selected hybridization probe.

In some embodiments of the kit, the plurality of hybridization probes can be combined in the same composition.

In any of the preceding embodiments, the kit can comprise a plurality of interfering agents, wherein each of the interfering agents is separate from the composition comprising the plurality of hybridization probes.

In any of the preceding embodiments, the kit can comprise multiple pluralities of hybridization probes, wherein each plurality of hybridization probes corresponds to a single round of sequential decoding.

In any of the preceding embodiments, the kit can further comprise one or more separate compositions comprising hybridization probes corresponding to one or more interfering agents of the kit.

In any of the preceding embodiments, each hybridization probe of the plurality can comprise an overhang region comprising a detection hybridization region specific for one of a set of detection probes, wherein each detection probe of the set corresponds to a different detectable label or the absence of a label.

In any of the preceding embodiments, each overhang region can further comprise a specific sequence corresponding to the recognition sequence of the hybridization probe.

In some embodiments, the interfering agent can comprise a quencher moiety, wherein the interfering agent is capable of hybridizing to the specific sequence of a selected hybridization probe.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show schematics comparing a reference in situ sequencing-by-hybridization (SBH ISS) round (FIG. 1A) and an exemplary SBH ISS with an interfering agent (antidote “anti-Target 2”) (FIG. 1B).

FIG. 2 shows two fluorescence microscopy images of a colon tissue section undergoing in situ sequencing-by-hybridization, showing all rolling circle products (RCPs) produced by rolling circle amplification (RCA) reactions (left) and all nuclei (right).

FIGS. 3A-3B show representative fluorescence microscopy images and decoding cycles of an exemplary SBH ISS reaction.

FIG. 4 shows representative fluorescence microscopy images of a reference SBH ISS round (left) and an exemplary SBH ISS round with antidote (right).

FIG. 5 shows representative fluorescence microscopy images of seven decoding cycles of an representative ISS SBH method with antidote probes (AD) and one “dote” round.

FIG. 6 shows representative fluorescence microscopy images from an exemplary “dote” round.

FIG. 7A provides a schematic showing reference sequential decoding rounds (in the absence of interfering agent), wherein signals associated with analyte 4 is highly abundant in the sample and has a dominant signal, preventing detection and/or resolution of other overlapping signals. Decoding of the dominant signal code sequence for analyte 4 enables identification of the highly abundant analyte and its selection for silencing using an interfering agent (antidote).

FIG. 7B provides a schematic depicting sequential decoding of the analytes of FIG. 7A using the same pools of L-probes and detection probes, but with the addition of an interfering agent designed to interfere with hybridization and/or signal generation and/or detection of the probes corresponding to analyte 4. The same interfering agent can be used in each decoding round.

FIG. 8A depicts an exemplary workflow for a method disclosed herein.

FIG. 8B shows a schematic of an exemplary “dote” probe for detecting analyte 4, and a schematic representation of analyte 4 detection in a “dote” round.

FIG. 9 provides a schematic of exemplary probe sets for detecting a plurality of target analytes in a sample by multiple sequential rounds of decoding. The vertical columns indicate the probe mixtures that can be contacted with the sample in each cycle. It will be appreciated that the same sequence of hybridization probe and detection probe mixtures or pools can be used regardless of which target nucleic acid sequence is highly abundant in the sample, and the interfering agent can be custom-selected to block detection of the highly abundant target nucleic acid sequence. The same interfering agent can be used in multiple cycles of sequential decoding, until the less abundant target nucleic acid sequences have been decoded.

FIGS. 10A-10B depict interfering agent designs for displacing a selected probe (also referred to as a second probe).

FIGS. 11A-11B depict interfering agent designs comprising quencher moieties.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In multiplex assays, such as multiplexed in situ gene expression and/or protein analysis, signal crowding problems can arise when there are a large number of signals to be detected. For example, combinatorial barcoding approaches are often used to encode a large number of analytes, and optical signals (e.g., spots in fluorescent microscopy) from the analytes or probes bound thereto are detected and decoded. Because the number of spots detected typically scales with the number of analytes assayed, a sample can become crowded with signal spots that overlap with each other thereby making resolution of individual spots difficult and reducing overall assay sensitivity. Thus, spatial overlap (e.g., optical crowding) may limit the ability to multiplex in assays such as microscopy-based nucleic acid hybridization or sequencing assays.

In some aspects, signal crowding may arise when one or more of the signals being detected are significantly stronger (e.g., have a significantly larger amplitude) than other signal(s). For example, in the same microscope field of view, one or more fluorescent spots may be significantly stronger than other spots including neighboring spots. In other aspects, signal crowding may arise when one or more of the signals being detected are in close proximity (e.g., overlapping to some degree) with other signal(s), such as overlapping signals observed in the same microscope field of view. When too many signals (e.g., “spots”) are present or when the amplitude of a signal is significantly greater than that of another signal, it can be difficult to accurately and reliably detect all of the signals in the same field of view and/or in the same detection channel (e.g., the same fluorescent channel). In some examples, this can cause weaker (e.g., lower amplitude) or overlapping signals to be “crowded out” or masked, which ultimately leads to information from the system being lost. In such circumstances, the effective dynamic range of the detection assay can be reduced.

The present disclosure provides methods of detecting multiple analytes (e.g., target nucleic acid sequences or proteins) in a sample so as to reduce signal crowding contributed by detection of the one or more analytes. In some embodiments, an analyte known to cause (or suspected of contributing to) signal crowding in a sample is selected prior to a given cycle, step, or round of a method disclosed herein, and a detection probe for the selected analyte, a secondary (or higher order) probe specific to a sequence in, e.g., the detection probe or an amplified product thereof, and/or a detection agent (e.g., a fluorescently labelled detection oligo) for the analyte or probe(s) may be manipulated in the cycle, step, or round. In some embodiments, a plurality of analytes known to cause or suspected of contributing to signal crowding are selected. In some embodiments, hybridization between the selected analyte(s), probe(s) thereto, and/or detection agent(s) for the analyte(s) or probe(s) is manipulated, e.g., by using an agent that interferes with the hybridization. In some embodiments, the presence/absence or amount of probe(s) to the selected analyte(s) and/or detection agent(s) for the analyte(s) or probe(s) is manipulated using a method disclosed herein.

In some embodiments, one or more analytes to be detected, probe(s) thereto, and/or detection agent(s) for the analyte(s) or probe(s) are manipulated in a cycle, step, or round of the method, wherein such analytes are not pre-selected based on the knowledge or suspicion that they may contribute to signal crowding, but are designated to be manipulated in a given cycle, step, or round in a random and/or combinatorial manner (e.g., from among multiple analytes to be detected in a sample). The multiple analytes to be detected may be pre-selected, e.g., targeted for analysis in a multiplex assay, but the designation of one or more of these analytes to be manipulated in a given cycle, step, or round is random and/or as part of a combinatorial scheme. In some embodiments, a plurality of analytes are randomly and/or combinatorially designated for manipulation in a cycle, step, or round of the method. In some embodiments, hybridization between the designated analyte(s), probe(s) thereto, and/or detection agent(s) for the analyte(s) or probe(s) is manipulated, e.g., by using an agent that interferes with the hybridization.

In some embodiments, signals from one or more analytes to be detected, probe(s) thereto, and/or detection agent(s) for the analyte(s) or probe(s) are modified. These signals may be prevented from being generated and/or detected, or may be detected but reduced in amplitude, e.g., through manipulation of the analyte/probe binding (e.g., hybridization) and/or the probe/detection agent binding (e.g., hybridization). These signals may be also be generated and/or detected over an increased time period, e.g., by distributing signals that can be generated and/or detected in one cycle, step, or round among a plurality of cycles, steps, or rounds. Different analytes may be detected at different rounds or cycles, steps, or rounds of the method, and this may be achieved in a number of ways. In some embodiments, a method disclosed herein reduces the number of signals that are generated and/detected from the sample at a given time, or in a given cycle, step, or round, and therefore reduces signal crowding. In some embodiments, a method disclosed herein reduces signal(s) or the strengths of signal(s) indicative of certain analyte(s) that are generated and/or detected from the sample at a given time, or in a given cycle, step, or round. These signals (e.g., indicative of highly expressed genes in a sample), if not reduced, may crowd out or mask signals indicative of other analyte(s). In some examples, these aspects of the present disclosure are referred to as the “antidote” approach. The present disclosure allows for more of the signals to be resolved, and therefore more of the analytes (e.g., target nucleic acid sequences) to be detected in a sample.

### II. Samples, Analytes, and Target Sequences

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprise one or more post-fixing (also referred to as postfixation) steps. In some embodiments, one or more post-fixing step is performed after contacting a sample with a polynucleotide disclosed herein, e.g., one or more probes such as a hybridization probe and/or a circular probe or circularizable probe or probe set (such as a padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample are known in the art, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method known in the art. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods are known in the art. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes of varying abundances (e.g., to enable visualization of lowly abundant and highly abundant analytes in a sample). In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

(a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. In some embodiments, an interfering agent (antidote probe) described herein interferes with hybridization of a barcoded probe or probe set to a target nucleic acid sequence in or associated with an analyte. For example, an interfering agent can hybridize to one or more sequences of the barcoded probe or probe set and prevent their hybridization to the target nucleic acid sequence, or can hybridize to the target nucleic acid sequence.

(b) Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof

In some embodiments, provided herein is a probe or probe set capable of DNA-templated ligation, such as from a cDNA molecule. See, e.g., U.S. Pat. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety. In some embodiments, an interfering agent (antidote probe) described herein interferes with ligation of a barcoded probe or probe set to form a circularized template, thereby interfering with generation of a rolling circle amplification product corresponding to a selected target nucleic acid sequence.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. This selectively reduces the concentration of annealed mismatched substrates (expected to have a slightly lower Tm around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, a product is a primer extension product of an analyte, a labelling agent, a probe or probe set bound to the analyte (e.g., a padlock probe bound to genomic DNA, mRNA, or cDNA), or a probe or probe set bound to the labelling agent (e.g., a padlock probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) are known in the art such as linear RCA, a branched RCA, a dendritic RCA, or any combination thereof (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-119, 2000; Faruqi et al, BMC Genomics 2:4, 2000; Nallur et al, Nucl. Acids Res. 29:el 18, 2001; Dean et al. Genome Res. 11: 1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, U.S. Pat. No. 10,494,662, WO 2017/079406, U.S. Pat. No. 10,266,888, US 2016/0024555, US 2018/0251833 and US 2017/0219465, the contents of each of which are herein incorporated by reference in their entirety. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (i.e. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein (e.g., a hybridization probe) may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe (e.g., a hybridization probe). The exogenously added nucleic acid probe may be optionally ligated to a cellular nucleic acid molecule or another exogenous nucleic acid molecule. In other examples, a product comprising a target sequence for a probe disclosed herein (e.g., a hybridization probe) may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein (e.g., a hybridization probe) may a probe hybridizing to an RCP. The probe may comprise an overhang that does not hybridize to the RCP but hybridizes to another probe (e.g., a hybridization probe). The probe may be optionally ligated to a cellular nucleic acid molecule or another probe, e.g., an anchor probe that hybridize to the RCP.

C. Target Sequences

A target sequence for a probe disclosed herein (e.g., a hybridization probe) may be comprised in any analyte disclose herein, including an endogenous analyte (e.g., a viral or cellular nucleic acid), a labelling agent, or a product of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub. 20210164039, which are hereby incorporated by reference in their entirety.

### III. Signal Crowding

In some embodiments, the present disclosure addresses signal crowding in methods that involve detecting nucleic acid sequences (either as the target analytes or as the labels or reporters for one or more target analytes, such as one or more target proteins), including in situ assays that detect the localization of analytes in sample. There are a number of situations in which it is desired to detect several different analytes in a sample simultaneously, for example when detecting the expression of different genes in situ in a sample, where there can be a wide range of different expression levels. In some embodiments, nucleic acid molecules are detected as target analytes in situ in a sample. In some embodiments, nucleic acid molecules are detected as reporters for other, non-nucleic acid analytes, including for example proteins, or indeed as a reporter, or signal amplifier, for a nucleic acid analyte. Thus, in a detection assay for such an analyte, nucleic acid molecules may be used, for example as a tag or reporter for an antibody or other affinity-binder-based probe (e.g. in immunoPCR or immunoRCA), or generated, for example by ligation or extension in a proximity probe-based assay. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions. The nucleic acid molecule may be present in an amount which reflects the level of the analyte and may be detected as a “proxy” for the target analyte. Suitable methods for detecting multiple nucleic acid sequences in a sample are well known in the art, including the use of hybridization probes and sequencing-by-hybridization.

In some embodiments, a method disclosed herein comprises labelling analytes to be detected (either directly or indirectly) with detectable labels, using hybridization probes for example, and then detecting signals from those labels in order to identify the nucleic acid sequences. In some embodiments, some of the target nucleic acid sequences are present in the sample at significantly higher or lower concentrations than the other target nucleic acid sequences. If a particular target nucleic acid sequence is present in the sample at a high concentration, then a large number of hybridization probes will be bound to that target nucleic acid sequence and a large number of signals will be generated. In some embodiments, multiple signals are generated and detected concurrently, and the number of signals that are generated from each target nucleic acid sequence is related to the amount of that target nucleic acid sequence which is present in the sample. Accordingly, signals from target nucleic acid sequences which are present in high concentrations or in close proximity to signals from other target nucleic acid sequences may overcrowd and mask signals from the target nucleic acid sequences. In some embodiments, a method disclosed herein prevents and/or ameliorates signal crowding in multiplex assays where it is desired to detect a number of different nucleotide sequences, regardless of the means by which the sequences are labelled, and the type of labelling that is used (e.g. optical signals, radioactive signals, etc.). The present disclosure is particularly useful where a number of different signals are being generated simultaneously in close proximity.

In some embodiments, a method disclosed herein comprises detecting and identifying RNA transcripts in a given cell, in order to analyze the gene expression of that cell. In some embodiments, a method disclosed herein comprises labelling the RNA transcripts (or one or more primary or higher order probes bound thereto) with fluorescently labelled probes. The signals from the fluorescent labels can then be visualized in order to determine which RNA transcripts are present in a given cell of, e.g., a tissue sample. This can also be used to provide information on the location and the relative quantities of different RNA transcripts (and therefore the location and relative levels of expression of the corresponding genes). If a particular gene (or genes) is significantly overexpressed, a large number of RNA transcripts corresponding to that gene will be present in the sample, and thus a large number of fluorescent signals indicating the presence of that RNA transcript will be generated. At a certain point, the signal density will be such that at least some individual signals cannot be resolved using conventional fluorescence microscopy, thereby inhibiting or even preventing the detection of signals from other RNA transcripts corresponding to genes which are expressed at a lower level or that physically overlap or are otherwise in close proximity in the sample (either in 2D or 3D space), which leads to a loss of information and an inaccurate picture of gene expression. It will be understood that this problem can occur in many other nucleic acid detection methods. In some aspects, the present disclosure provides a method of detecting multiple target nucleic acid sequences in a sample wherein signal crowding is reduced.

In some embodiments, the methods provided in this disclosure are for use in the multiplexed detection of analytes (such as nucleic acids), that is, for the detection of multiple target analytes in a sample, e.g., one or more tissue samples such as a single tissue section or a series of tissue sections. In some embodiments, the methods use hybridization probes, whilst reducing signal crowding from said hybridization probes. In some embodiments, the methods provided herein comprise sequencing-by-hybridization (SBH) for detecting nucleic acid sequences in a sample, including multiplex SBH for detecting different target nucleic acid sequences (e.g., labels or reporters for one or more target analytes), with a wide range of distribution and abundance simultaneously in a sample. In some embodiments, the methods provided herein address signal crowding issues due to signals indicative of target nucleic acid sequences present in high concentrations and/or close proximity that may mask and/or overcrowd other signals.

In some aspects, signal overcrowding may prevent signals relating to the target nucleic acid sequences from being generated, detected, or otherwise distinguished from other signals in the sample. For example, if the hybridization probes cannot successfully hybridize to their cognate target nucleic acid sequences due to steric hindrances, or if detection probes cannot hybridize to the hybridization probes, then signals will not be generated and thus the target nucleic acid sequences will not be detected. This may be referred to as steric crowding. Alternatively, it may be that signals are properly generated from all of the target nucleic acid sequences, but that so many signals are generated, either in a particular area of the sample or in the sample as a whole (e.g., the signal density is too large), that not all of the signals can be properly detected and resolved. Where the signals are detected by optical means, this may be referred to as optical crowding, and the present methods are particularly suited to resolving, or reducing, optical crowding. In some aspects, by “optical means” is meant that the signals are detected visually, or by visual means, namely that the signals are visualized. Thus, in some instances, the signals that are generated involve detection of light or other visually detectable electromagnetic radiation (such as fluorescence). In some aspects, the signals may be optical signals, visual signals, or visually detectable signals. The signals may be detected by sight, typically after magnification, but more typically they are detected and analyzed in an automated system for the detection of the signals.

In some aspects, the signals may be detected by microscopy. In some aspects, an image may be generated in which the signals may be seen and detected, for example an image of the field of view of a microscope, or an image obtained from a camera. The signals may be detected by imaging, more particularly by imaging the sample or a part or reaction mixture thereof. By way of example, signals in an image may be detected as “spots” which can be seen in the image. In some aspects, a signal may be seen as a spot in an image. In some aspects, optical crowding occurs when individual spots cannot be resolved, or distinguished from one another, for example when they overlap, or mask one another. By reducing the number of spots using the methods herein, such that individual spots, or signals, can be resolved, optical crowding can be reduced. In some aspects, the present methods optically de-crowd the signals.

In some aspects, the reduction in signal crowding associated with the present methods may be considered to be a reduction relative to the level of signal crowding which would occur in a method which did not comprise steps to reduce signal crowding, for example, without the use of antidote probes described herein.

In some aspects, the methods herein involve reducing the number of signals that are detected in a detection step of the method. This is achieved in different ways in the different methods, to prevent or block a signal from being generated from certain targets (e.g., abundant or highly expressed targets in a sample) in a given cycle of detection.

### IV. Antidote Approaches

### A. Methods and Exemplary Workflows

In some embodiments, provided herein is a method for analyte detection, comprising contacting a sample, a first probe, a second probe, and an interfering agent (also referred to as an “antidote probe” or “antidote”) with one another in any suitable order. In some embodiments, the sample is contacted with the first and second probes and the interfering agent in the same reaction volume. In other embodiments, the sample is contacted with the interfering agent before or after the sample is contacted with the first and second probes. In some embodiments, the first probe (e.g., a hybridization probe or a circular probe or circularizable probe or probe set) is capable of directly or indirectly hybridizing to a first target nucleic acid sequence (e.g., a first reporter sequence or barcode associated with a given analyte), the second probe (e.g., a hybridization probe or a circularizable probe or probe set) is capable of directly or indirectly hybridizing to a second target nucleic acid sequence (e.g., a second reporter sequence or barcode associated with a given analyte). In some embodiments, the first and second target nucleic acid sequences are different, and hybridization of the first probe to the first target nucleic acid sequence is not interfered by the interfering agent, whereas hybridization of the second probe to the second target nucleic acid sequence is interfered by the interfering agent.

In some embodiments, the method comprises detecting a signal indicative of the hybridization of the first probe to the first target nucleic acid sequence in the sample, whereas a signal indicative of the hybridization of the second probe to the second target nucleic acid sequence in the sample is not detected or is detected at a lower level compared to a reference signal detected without the interfering agent interfering with hybridization of the second probe to the second target nucleic acid sequence, thereby detecting the first target nucleic acid sequence in the sample.

In some embodiments (e.g., wherein the first and second probes are circularizable probes or probe sets), the method comprises detecting a signal indicative of the hybridization and of the first probe to the first target nucleic acid sequence in the sample and the ligation (e.g., circularization) of the first probe, whereas a signal indicative of the hybridization of the second probe to the second target nucleic acid sequence in the sample and the ligation (e.g., circularization) of the second probe is not detected or is detected at a lower level compared to a reference signal detected without the interfering agent interfering with hybridization of the second probe to the second target nucleic acid sequence, thereby detecting the first target nucleic acid sequence in the sample.

In any of the embodiments herein, the interfering agent can be or comprise one or more probes, termed herein “antidote probes” in some examples, that prevent signals from being generated and/or detected or that reduce the amplitude of signals indicative of certain (e.g., selected) target nucleic acid sequences (e.g., reporter sequence(s) or barcode(s) associated with a given analyte). In some aspects, an antidote probe can be viewed as a blocking probe, which blocks, inhibits, or prevents a hybridization probe from functioning to detect its target and/or a detection probe from functioning to detect the hybridization probe. In some embodiments, this may be achieved by blocking or reducing the binding of the hybridization probe to its target (e.g., by allowing an antidote probe to hybridize to the hybridization probe or by allowing an antidote probe to hybridize to the target). In some embodiments, an antidote probe can comprise a quenching moiety that inhibits the signal of a fluorescent moiety associated with the hybridization probe to which the antidote probe is hybridized, thereby preventing a hybridization probe from functioning to detect its target and/or a detection probe from functioning to detect the hybridization probe.

In some embodiments, the method comprises blocking or reducing the binding of one or more detection probes (e.g., fluorescently labelled oligos) to the hybridization probe while binding between the hybridization probe and its target is not blocked or reduced. In some embodiments, the antidote probe hybridizes to a detection probe (e.g., fluorescently labelled oligo). In some embodiments, the method comprises allowing an antidote probe to hybridize to a hybridization probe at a sequence outside of the target-binding sequence of the hybridization probe (e.g., an overhang of the hybridization probe hybridized to its target). In some aspects, an antidote probe may be termed a “silencer probe,” as it can be seen to function to silence a signal indicative of an analyte, e.g., a signal from a hybridization probe corresponding to the analyte. In some aspects, the methods for removing the signal indicative of the analyte is reversible and/or temporary. For example, the interfering agent can be removed and the signal associated with an analyte can be detected after the removal.

In one aspect, provided herein is a method of detecting multiple target nucleic acid sequences in a sample (e.g., multiple reporter sequences or barcodes associated with analytes in a sample), wherein the target nucleic acid sequences are detected by hybridization probes which hybridize to the target nucleic acid sequences and provide detectable signals that allow the target nucleic acid sequences to be identified and detected, said method comprising: (a) providing a plurality of hybridization probes comprising different hybridization probes each specific for a different target nucleic acid sequence, wherein each hybridization probe has a recognition sequence complementary to a sequence within its target nucleic acid sequence, and is capable of giving rise to a signal by means of which it can be detected; (b) further providing at least one antidote probe for at least one selected hybridization probe specific for a selected target nucleic acid sequence; (c) contacting the sample with the plurality of hybridization probes of (a) and the antidote probe(s) of (b), allowing the antidote probe(s) to hybridize to the selected hybridization probe(s) or the selected target nucleic acid sequence(s), and allowing the hybridization probes to hybridize to target nucleic acid sequences that are present in the sample; (d) detecting a signal from each hybridization probe which has hybridized to its target sequence, wherein signals are not detected from the selected target nucleic acid sequences for which antidote probes were provided in (b); (e) identifying target nucleic acid sequences from the signals detected in step (d), and thereby detecting those target nucleic acid sequences in the sample.

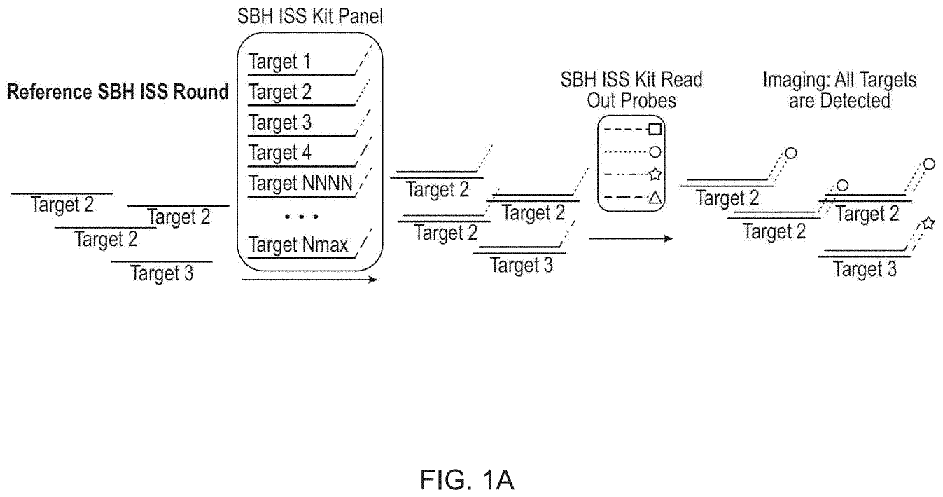

In some embodiments, in the step of providing at least one antidote probe for at least one selected hybridization probe specific for a selected target nucleic acid sequence (step (b)), each antidote probe comprises a sequence complementary to a sequence within the selected hybridization probe and is capable of hybridizing to it to form a hybridization probe-antidote probe complex which is not capable of providing a signal that allows the selected target nucleic acid sequence to be detected (e.g., the complex is not able to hybridize to the corresponding target nucleic acid sequence, as shown in FIG. 1B, Target 2+anti-Target 2 complex) In some embodiments, each antidote probe comprises a sequence complementary to a sequence within the selected target nucleic acid sequence and is capable of hybridizing to it to form a target nucleic acid sequence-antidote probe complex (e.g., preventing or reducing hybridization of the hybridization probe to the target) which is not capable of providing a signal which allows the selected target nucleic acid sequence to be detected. In some embodiments, a first antidote probe hybridizes to a first selected hybridization probe to form a hybridization probe-antidote probe complex which is not capable of providing a signal that allows the corresponding first selected target nucleic acid sequence to be detected, while a second antidote probe hybridizes to a second selected target nucleic acid sequence to form a target nucleic acid sequence-antidote probe complex (e.g., preventing or reducing hybridization of the corresponding second selected hybridization probe to the target) that is not capable of providing a signal which allows the second selected target nucleic acid sequence to be detected (e.g., because the corresponding hybridization probe cannot hybridize to the second selected target nucleic acid sequence).

In some embodiments, the method reduces signal crowding from said hybridization probes because signals are not detected from the selected target nucleic acid sequences, for example, due to the selected target nucleic acid sequences (e.g., barcode(s) associated with the selected analyte) being more abundant than other target nucleic acid sequences in the sample.

In some embodiments, the presence of the antidote probes prevents signals from being detected from the selected target nucleic acid sequences of (b), which reduces signal crowding and therefore allows the signals from other, non-selected, target nucleic acid sequences to be resolved.

In some embodiments, the presence or absence of a detectable signal, or more particularly the presence or absence of a detectable label, is analyzed. For example, a hybridization probe can be distinguished from other, labelled, hybridization probes by the absence of signal. Accordingly, in an embodiment, hybridization probes are directly or indirectly labelled with a detectable label which gives rise to a signal, optionally wherein a hybridization probe in the plurality is not labelled with a detectable label and is detected by absence of a signal. The step of detecting a signal from each hybridization probe which has hybridized to its target sequence (step (d)) can comprise detecting a signal from the detectable label (e.g., on a detection oligo that hybridizes to a hybridization probe) of each hybridization probe that has hybridized to its target sequence, and optionally detecting absence of signal where a hybridization probe is not labelled or is prevented from hybridizing to its target in the sample.

In some embodiments, the target nucleic acid sequences for which an antidote probe or antidote probes are provided can be selected based on their relative abundance in the sample. For example, sequences which are present at high levels (high abundance), and which do or may lead to signal crowding, can be “removed” from the detection in step (d). The target nucleic acid sequences which are selected (in an optional selecting step, (b)) may be selected on the basis of their abundance in the sample or based on the relative levels of the target nucleic acid sequences which are present, or which are to be detected, in the sample. In some embodiments, the selected target nucleic acid sequences may be present in the sample in an increased amount relative to other target sequences which are present in the sample, or which are to be detected in the sample. For example, the target nucleic sequences may be sequences of genes in the sample, and certain genes may be more highly expressed in the sample than others, and their corresponding target sequences may be selected to be blocked using an interfering agent (antidote) in step (b). In some embodiments, the analyte may be another type of molecule present in the sample, e.g. a protein, which is detected by an assay involving a reporter nucleic acid sequence which is represents the target nucleic acid sequence, and which is present or generated in the sample in an amount which reflects, or which is indicative of, or proportional to, the amount of target analyte present in the sample.

The selection of target nucleic acid sequences (i.e., target nucleic acid sequences for blocking by an interfering agent) may be predetermined, for example based on what is known about the levels of the target analytes in samples, or may be determined empirically. Accordingly, the method may comprise preceding steps of detecting target nucleic acid sequences in the sample using the plurality of hybridization probes of step (a) and determining the presence in the sample of target nucleic acid sequences which give rise to signals which crowd out signals from other target nucleic acid sequences in the sample, and wherein those target nucleic acid sequences are selected for step (b). Alternatively, the method may not comprise a step of selecting the target nucleic acid sequences for blocking (step (b)), as said sequences are pre-selected based on what is known about the levels of target analytes in a given sample.

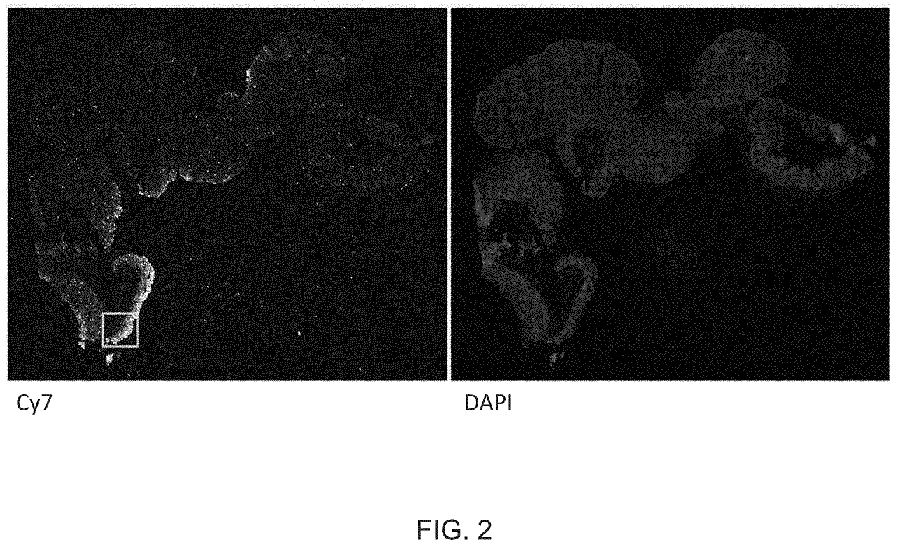

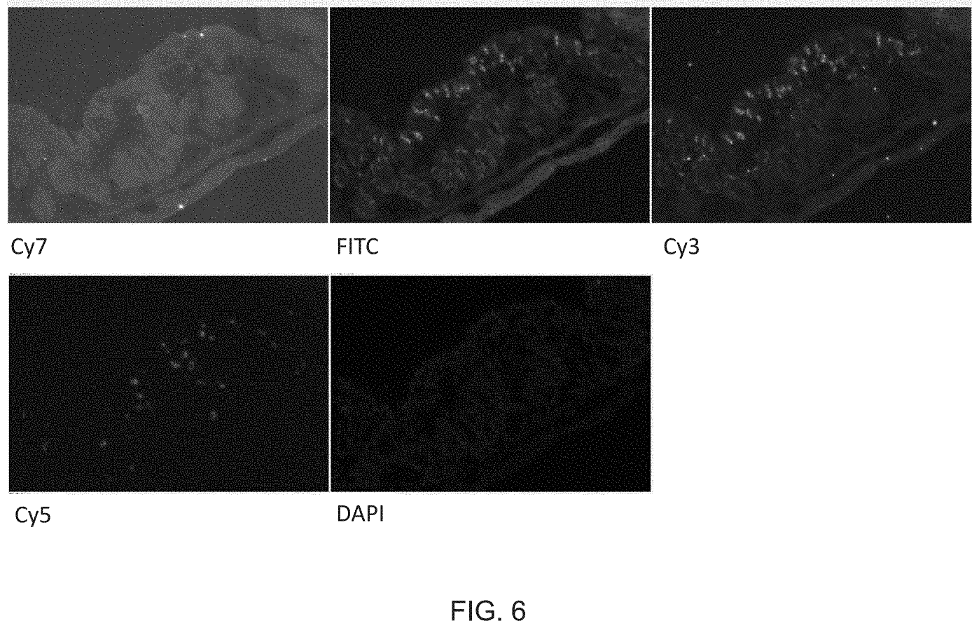

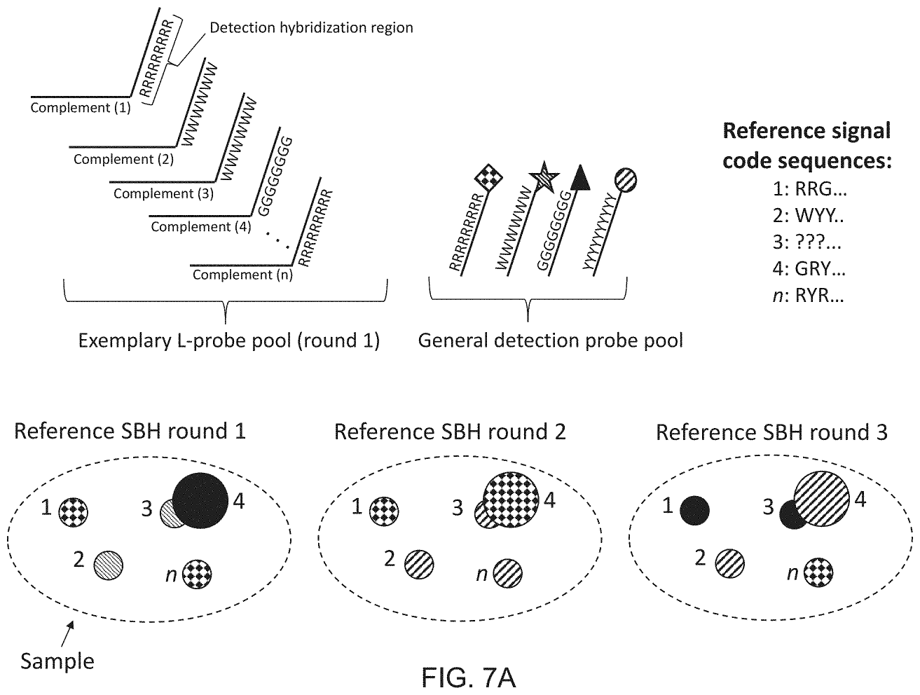

In some embodiments, a method for selection of target nucleic acid sequences for blocking by an interfering agent (antidote) comprises detecting or identifying a dominant, or particularly large, signal in a sample, or a bulk signal or signal change in the sample. In the context of a sequential combinatorial labelling scheme for example, it may be possible to detect and see bulk signal changes in the sample, from cycle to cycle, by simply visually following the cycles to detect signal changes, e.g. color changes. Target nucleic acid sequences which are particularly abundant and the signals from which may be crowding out other signals, may be detected because a large amount of signal is detected therefrom, causing the sample, or an area of the sample in an image for example, to change signal (e.g. color), the same way from cycle to cycle. Example 1 provides results demonstrating identification and selection of target nucleic acid sequences for blocking by an interfering agent by detecting highly abundant signal changes crowding out other signals in particular areas of the sample (see FIGS. 3A-3B). Similarly, FIG. 7A provides a schematic example of how a target nucleic acid sequence associated with a dominant signal (spot 4) can be decoded in a sample.

In some embodiments, following the identification of the non-selected target nucleic acid sequences from one or more antidote detection cycles (step (e)), further detection steps may be carried out in order to detect the selected target nucleic acid sequences (e.g., the target nucleic acid sequences for which the signal was reduced or eliminated using the “antidote”). In some embodiments, the method further comprises: (f) removing the hybridization probes from the target nucleic acid sequences; (g) contacting the sample with one or more further hybridization probes, each specific for a selected target nucleic acid sequence of for which antidote probes were provided in the previous round, wherein each further hybridization probe has a recognition sequence complementary to a sequence within its selected target nucleic acid sequence, and is capable of giving rise to a signal by means of which it can be detected, and allowing the further hybridization probes to hybridize to the selected target nucleic acid sequences; (h) detecting a signal from each further hybridization probe which has hybridized to its target sequence; (i) identifying the selected target nucleic acid sequences from the signals detected for the selected target nucleic acid sequences in step (h), thereby detecting those selected target nucleic acid sequences that are present in the sample. In some embodiments, this use of one or more additional rounds of hybridization and detection with further hybridization probes allows for signals from the selected target nucleic acid sequences of (b) (e.g., target nucleic acid sequences removed using the “antidote”) to be detected separately from those from the non-selected target nucleic acid sequences (e.g., in an environment with less signal crowding, or where less abundant target sequences are not a concern), such that these signals can be detected and/or resolved and the corresponding nucleic acid sequences can be identified. In some examples, the further hybridization probes are termed “dote” probes, and the selected target nucleic acid sequence(s) may be detected in one or more “dote” cycles or rounds. It will be understood that one or more dote cycles or rounds can be performed before, in between, or after one or more “antidote” cycles or rounds.

As noted above, problems of signal crowding typically arise in methods which involve significant degrees of multiplexing (e.g., methods which involve detecting large numbers of different target nucleic acid sequences simultaneously). To facilitate multiplexing (e.g., to be able to distinguish between many different target nucleic acid sequences), these methods often involve combinatorial labelling schemes and multiple decoding cycles, wherein each target nucleic acid sequence is assigned a unique signal code sequence comprised of several individual signal codes in a specific order, and one signal (corresponding to one signal code) is detected for each target nucleic acid sequence in each decoding cycle. The individual signals detected for each target nucleic acid sequence are recorded, and putative signal code sequences can be built up over time with each decoding cycle, until the full, or sufficient, signal code sequences have been detected and the target nucleic acid sequences can be identified. For example, in some embodiments, a signal code sequence can be designed to correspond to a barcode sequence, which is provided in the target nucleic acid sequence, and the barcode sequence can be detected in a SBH scheme using a series of hybridization probes in a specific order, each hybridization probe yielding an individual signal code of the signal code sequence. In some embodiments, a method disclosed herein can comprise sequential fluorescence hybridization of detectable probes, including detectably labeled probes (e.g., fluorophore conjugated oligonucleotides) and/or probes that are not detectably labeled per se but are capable of binding (e.g., via nucleic acid hybridization) and being detected by detectably labeled detection probes that hybridize to an overhang region of the probe (e.g., an L-shaped probe, comprising a single overhang region as shown in FIG. 7A, or a U-shaped probe comprising two overhang regions). Exemplary methods comprising sequential fluorescence hybridization of detectable probes are described in US 2019/0161796, US 2020/0224244, WO 2020/099640, US 2021/0340618, and WO 2021/138676, all of which are incorporated herein by reference. It will be appreciated that the methods described herein can be applied to interfere with the hybridization and/or detection of probes corresponding to selected target nucleic acids according to any of the aforementioned methods comprising sequential fluorescence hybridization.

FIGS. 1A-1B shows a schematic which highlights a difference between a reference in situ sequencing-by-hybridization (SBH ISS) round (FIG. 1A) and an exemplary method involving the use of antidote probes disclosed herein (FIG. 1B). Different target sequences (Targets 1−Nmax) may be barcode sequences in RCPs, which can be generated from barcoded padlock probes (or other circularizable probe or probe set) where the barcodes correspond to the analytes (e.g., RNA or cDNA) that the barcode padlock probe hybridizes to in a sample. In the reference round, Targets 2 and 3 are present in the sample and Target 2 is more abundant than Target 3. A SBH ISS kit panel comprising a plurality of hybridization probes targeting the barcode sequences in the RCPs may be contacted with the sample, each hybridization probe comprising a target nucleic acid recognition sequence and an overhang sequence. A plurality of read out probes (e.g., fluorescently labeled detection oligos) are contacted with the sample in sequential cycles to hybridize to the overhang sequences of the hybridization probes, thereby detecting the target sequences in the sample. In the reference round, signals from read out probes indicative of Target 2 is strong and may crowd out and/or overlap with the signals of Target 3.

In the method involving the use of antidote probes, in an antidote cycle, an antidote probe (anti-Target 2) is added which is complementary to the hybridization probe which corresponds to Target 2 in FIG. 1B. This antidote probe hybridizes to its cognate hybridization probe, thus forming a hybridization probe-antidote probe complex which cannot bind to the target nucleic acid sequence (Target 2). As a result, no signal (or a reduced signal) indicative of Target 2 is generated, allowing signals for Target 3 to be better resolved. The method may comprise one or more additional antidote cycles, each with antidote probe(s) for one or more targets, e.g., one or more of Targets 1−Nmax (including Target 2 which has been targeted in an antidote cycle). The method may further comprise one or more dote cycles before, after, or in between any of the antidote cycles, wherein each dote cycle uses hybridization probes for one or more targets for which probe hybridization and/or signal detection was interfered (i.e., eliminated or reduced) in the antidote cycles.

FIG. 7A provides a schematic illustrating a method of sequential decoding without antidote probes to determine reference signal code sequences corresponding to target analytes in a sample. In the illustrated example, target nucleic acid sequences 1, 2, 3, 4, and n are decoded. In this example, target nucleic acid sequence 4 corresponds to a highly abundant analyte in the sample, as indicated by the larger size of spot 4 in the sample. The signal for spot 4 spatially overlaps with the signal for spot 3, preventing detection and decoding of the signal code sequence corresponding to target nucleic acid 3.

In some embodiments, the methods disclosed herein comprise providing a plurality of hybridization probes comprising probes specific for different target nucleic acid sequences (e.g., different reporter or barcode sequences associated with a given analyte) and at least one antidote probe disclosed herein. The plurality of hybridization probes may be a mixture of hybridization probes which are complementary to the target nucleic acid sequences which are to be detected, such as the exemplary L-probe pool depicted in the top panel of FIG. 7. Thus, a composition may be provided comprising such a plurality or mixture of multiple hybridization probes. In some embodiments, the plurality of hybridization probes comprises a different hybridization probe for each target nucleic acid sequence (e.g., barcode sequence) to be detected. In some embodiments, a mixture (also referred to as a pool or library) of hybridization probes is provided for each of multiple sequential decoding rounds or cycles. In some embodiments, a hybridization probe of the plurality comprises a recognition sequence that hybridizes to a sequence of the target nucleic acid sequence (e.g., the complement of the sequence of the target nucleic acid sequence), as shown in FIG. 7. In some embodiments, the hybridization probes in the mixture comprise individually comprise a detection hybridization region (also referred to as a reporter region) that does not hybridize to the target nucleic acid, wherein each detection hybridization region corresponds to a detection probe. In FIG. 7, this correspondence is indicated by the letters “R” for red, “W” for white, “G” for green, and “Y” for yellow. As shown in FIG. 7, the same or different detection hybridization regions can be used for multiple different hybridization probes for the mixture, and the same or different detection hybridization regions can be used in multiple cycles for the same target analyte. Exemplary signal code sequences in the absence of antidote (“reference” signal code sequences) for target nucleic acid sequences 1, 2, 3, 4, and n are shown in the upper right panel. In this example, the overlap of the highly abundant target nucleic acid sequence 4 signal with signal 3 prevents decoding of target nucleic acid sequence 3. Therefore, target nucleic acid sequence 4 is identified as the signal responsible for crowding and can be chosen as the selected target nucleic acid (also referred to as the second target nucleic acid herein) for interference by an interfering agent (antidote probe).

Depending on the degree to which signal crowding occurs, the number of unique analytes selected to be blocked by antidote probe(s) may be varied. If it is desired to detect a large number of different analytes (e.g., target nucleic acid sequences corresponding to the unique analytes) in a given sample, and thus it is necessary to detect a large number of signals simultaneously, antidote probes may be employed to block signals indicative of more unique analytes (e.g., abundant/highly expressed analytes in a sample), in order to reduce the number of signals which are generated in any one cycle. In some embodiments, the method may involve the use of antidote probes for more than one, such as 2, 3, 4, 5, 10, 15, 20 or more unique analytes in a sample. This may also depend on the nature of the sample, and the target nucleic acids that are being detected, e.g. on how many targets are present in the sample in high amounts. In some embodiments, the number of unique analytes to be blocked by antidote probes is less than or equal to the number of distinguishable signals (e.g., the number of distinct detection channels). When the number of unique selected analytes blocked by antidote probes is less than or equal to the number of available detection channels, the selected analytes can be detected in a single “dote” hybridization round once previous hybridization probes for a previous cycle have been removed from the target nucleic acid sequences. In cases where the number of unique selected analytes blocked by antidote probes is greater than the number of detection channels, multiple “dote” hybridization rounds may be required to identify and detect the selected analytes. In some embodiments, the method involves the use of antidote probes for no more than 2, 3, 4, 5, 10, 15, or 20 unique analytes in a sample. In some embodiments, the method involves the use of antidote probes for no more than 4 unique analytes in the sample.

In some embodiments, each hybridization probe comprises a recognition sequence which is complementary to a sequence within the corresponding target nucleic acid sequence. In some embodiments, the target nucleic acid sequences each comprise a distinct target nucleotide sequence (e.g., a reporter or barcode sequence associated with a particular analyte, such as the “Target” sequences in FIGS. 1A-1B or spots 1, 2, 3, 4, and n in FIGS. 7A-7B) which is recognized by the respective hybridization probe which is specific for that target nucleic acid sequence. In some aspects, the target nucleic acid sequence may thus be viewed as a binding site, or binding domain (or recognition site) for the hybridization probe. In some aspects, the hybridization probes and the target nucleotide sequences correspond to each other, or are cognate for each other. In some embodiments, a hybridization probe and a target are cognate for each other in that the hybridization probe corresponds to and is designed to bind to a target nucleotide sequence within a particular target nucleic acid sequence.

A composition or mixture comprising multiple hybridization probes, or alternatively put, a plurality of hybridization probes, such as the exemplary L-probe pool in FIG. 7A, may be considered to be a general or universal hybridization probe mix. In many multiplex methods, it is advantageous to have a consistent hybridization probe mix (or a consistent sequence of hybridization probe mixes, as would be used in multiple cycles of sequential decoding) which can be used to detect a range of target nucleic acid sequences in a number of different reactions, rather than to re-formulate a separate hybridization probe mix for each reaction. For example, as noted above, it may be desired to detect target nucleic acid sequences in order to analyze the gene expression or protein composition of a particular cell sample. If multiple cell or tissue samples are to be analyzed in order to compare gene expression across different cell types or tissue samples or sections, it may be easier, cheaper, and quicker to use a single hybridization probe mixture (or a single sequence of hybridization probe mixtures) for analyzing all of the cell samples, than to design a separate hybridization probe mixture (or sequence of hybridization probe mixtures) for each sample. However, it will be understood that across different reactions, different target nucleic acid sequences may be present in different quantities (e.g., differentially expressed RNAs) or in different spatial locations (e.g., in different cell or tissue types), and thus the particular sequences which may cause signal crowding problems may be different. Therefore, there is a need to eliminate or reduce the signals generated by selected hybridization probes in a mixture (corresponding to highly abundant analytes in a sample of interest) to allow visualization of both very lowly abundant and very highly abundant analytes (e.g., lowly expressed and highly expressed mRNA molecules) in the same sample while avoiding a need for a custom probe library. The present disclosure addresses this and other needs.

In some aspects, a method disclosed herein allows for specific hybridization probes within a larger mixture (such as a standardized mixture of probes, such as probes specific for a target nucleic acid) to be targeted with antidote probes so as to prevent or otherwise attenuate the signals which are responsible for signal crowding in each specific situation without the need to reformulate and/or otherwise change the standardized probe set. In other aspects, the methods disclosed herein allow for specific hybridization probes within a larger mixture to be blocked (by antidote probes) from hybridizing to their targets, thereby preventing or otherwise attenuating the signals which are responsible for signal crowding in each specific situation. Embodiments of the present disclosure can avoid the need to reformulate the hybridization probe mixture, e.g., by allowing the same hybridization probe mixture to be used in several different reactions (e.g., across different cellular or tissue samples). This is advantageous in a commercial setting, or indeed in any situation, where a single hybridization probe mix (or a single sequence of hybridization probe mixes) can be provided, and a separate solution for the problem of signal crowding can be provided in a specific case, where and as needed, in the form of an antidote probe or antidote probe mixture, which is bespoke, or tailored to address the particular problem at hand, namely to block those particular targets which are causing a signal crowding problem in a particular situation, e.g. for a particular sample, or detection reaction.

In one aspect, provided herein is a method of detecting multiple target nucleic acid sequences in a sample (e.g., multiple reporter sequences or barcodes associated with analytes in a sample), wherein the target nucleic acid sequences are detected by hybridization probes which hybridize to the target nucleic acid sequences and provide detectable signals that allow the target nucleic acid sequences to be identified and detected, said method comprising providing a plurality of hybridization probes comprising different hybridization probes each specific for a different target nucleic acid sequence, wherein each hybridization probe has a recognition sequence complementary to a sequence within its target nucleic acid sequence, and is capable of giving rise to a signal by means of which it can be detected, and the method further comprises providing at least one antidote probe for at least one selected hybridization probe/corresponding selected target nucleic acid (also referred to as a second hybridization probe and second target nucleic acid herein, respectively). In some embodiments, the selected target nucleic acid is pre-determined (e.g., a selected target nucleic acid with high abundance may be pre-determined for a given tissue type). In some embodiments, the selected target nucleic acid is identified by reference decoding in the absence of antidote, as shown in FIG. 7A.

In some embodiments, the method comprises a step (c) of contacting the sample containing the target nucleic acid sequences to be detected with the plurality of hybridization probes of step (a) and the at least one antidote probe identified for the selected target nucleic acid sequence (second target nucleic acid sequence) of step (b). The antidote probe is allowed to hybridize to the cognate selected hybridization probe, and the hybridization probes are allowed to hybridize to the target nucleic acid sequences, or the antidote probe is allowed to hybridize to the cognate target nucleic acid, and the hybridization probes are allowed to hybridize to the target nucleic acid sequences (if not blocked by the antidote probe). An example of sequential decoding with interfering agent(s) (antidote probe(s)) is shown in FIG. 7B. As shown in the top panel, the same hybridization probe mixture (L-probe pool) and general detection probe pool can be used as were used in FIG. 7A. In this case, however, in interfering agent (antidote probe) is added to the sample to interfere with detection of target nucleic acid 4. This reduces signal crowding in the sample and enables decoding of target nucleic acid 3, which was previously obscured by the target nucleic acid 4 signal. The same interfering agent(s) and general detection probe pools can be used in multiple sequential rounds of decoding.

In some embodiments, the method may comprise further steps (f) to (i) in order to identify and detect the target nucleic acid sequences for which detection was blocked using an interfering agent (antidote) in the previous steps (e.g., the target nucleic acid sequences which were selected in step (b)). In this regard, the method may further comprise a step (f) of removing the hybridization probes from the target nucleic acid sequences. The step of removing the hybridization probes may be done by any suitable means known in the art. This may involve the use of high temperature and/or chemical agents in order to denature or disrupt the hybrid formed between the target nucleic acid sequences and the hybridization probes. For example, the sample may be treated with formamide in order to remove the hybridization probes, e.g., along with reporter/detection probes hybridized to the hybridization probes.

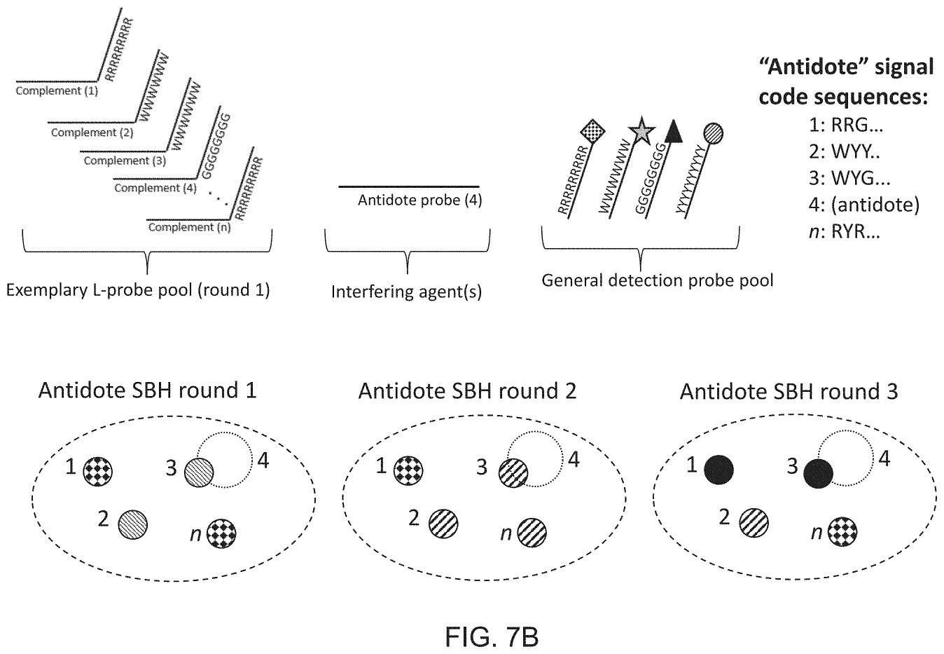

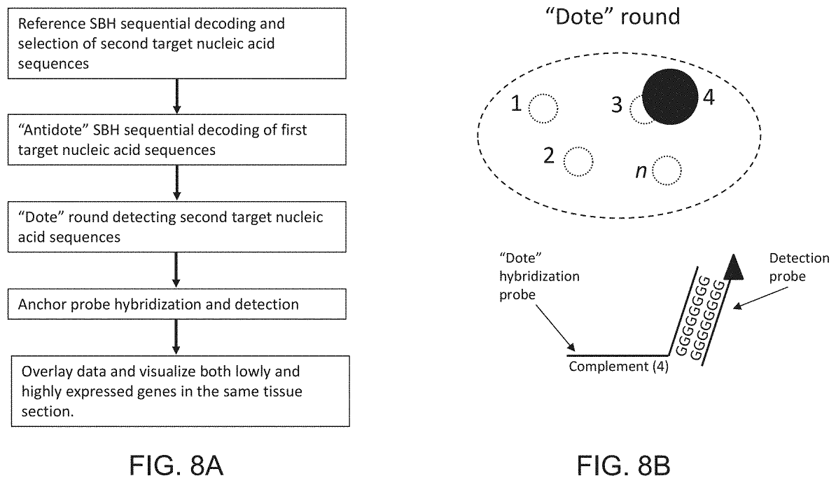

In some embodiments, once previous hybridization probes for a previous cycle have been removed from the target nucleic acid sequences, the method may comprise contacting the sample with one or more further (e.g., “dote”) hybridization probes, which are specific for one or more of the selected target nucleic acid sequences that have been targeted by antidote probes. In some embodiments, the further hybridization probes have the same structure as the hybridization probes provided prior to the antidote cycle(s), in that each further hybridization probe has a recognition sequence complementary to a sequence within its selected target nucleic acid sequence, and is capable of giving rise to a signal by means of which it may be detected. An exemplary “dote” hybridization probe is shown in FIG. 8B, wherein the hybridization probe comprises a recognition sequence (the complement of the target nucleic acid sequence 4) and a detection hybridization region specific for a detection probe. In some embodiments, a hybridization probe may be directly or indirectly labelled with a detectable label which gives rise to a signal, and optionally a hybridization probe may lack a detectable label, and may be detected by absence of detectable signal. Accordingly, the disclosures herein regarding the structure of the hybridization probes (including the structure and detection of the detectable labels), and their binding to the corresponding non-selected target nucleic acid sequences, apply equally in relation to the further hybridization probes and their binding to the corresponding selected target nucleic acid sequences.

In some embodiments, the further hybridization probes are allowed to hybridize to the selected target nucleic acid sequences. In some embodiments, the method may include a step of incubation to allow this hybridization to occur. Once the further hybridization probes have hybridized to the target nucleic acid sequences, the method may comprise a step (h) of detecting a signal from each hybridization probe, e.g. from the detectable label of each further hybridization probe that has hybridized to the corresponding target sequence, or in the case of an unlabeled hybridization probe, the absence of a detectable signal. Based on these signals, the identity of the selected (second) target nucleic acid sequences can be determined, and thus the selected target nucleic acid sequences within the sample can be detected in a step (i).

In some embodiments, in both the step of identifying non-selected (first) target nucleic acid sequences (step (e)) and the step of identifying the selected (second) target nucleic acid sequences (step (i)) of the method disclosed above, the signals detected from the detectable labels of the hybridization probes allow the corresponding target nucleic acid sequences to be identified. In some aspects, there is a link between the signals which are detected from the hybridization probes, and the identity of the target nucleic acid sequences. The system by which the signals which are detected from the hybridization probes encode the identity of the target nucleic acid sequences may vary depending on the number of target nucleic acid sequences which are to be detected. In some embodiments, each target nucleic acid sequence may be assigned a specific signal, and thus the signal detected from the hybridization probe may directly indicate the identity of the target nucleic acid sequence. However, where a large number of target nucleic acid sequences are to be detected simultaneously, e.g., where a high degree of multiplicity is necessary, and where signal crowding is likely to be a problem, the number of different signals available may be insufficient to assign each target nucleic acid sequence a unique signal, and thus a more complex system of coding the identity of the target nucleic acid sequences may be required. This can be a combinatorial labelling scheme as discussed above, and more particularly a sequential combinatorial labelling scheme, wherein a sequence of signals is generated and determined in sequential steps, or cycles.

In some embodiments, the target nucleotide sequence within each target nucleic acid sequence comprises a barcode sequence. This barcode sequence can be used to identify the target nucleic acid sequence. In some embodiments, the barcode sequence for each target nucleic acid sequence corresponds to a unique signal code sequence. That is, the barcode may be decoded (or put another way, read or identified) in a sequential series of cycles, wherein in each cycle a signal code of the signal code sequence is determined in turn, and in sequence. This signal code sequence is specific to the target nucleic acid sequence. Alternatively put, each target nucleic acid sequence is assigned a unique signal code sequence. This unique signal code sequence may be derived by interrogating the barcode sequence with hybridization probes capable of providing detectable signals in sequential decoding cycles, wherein each cycle yields a signal from a hybridization probe which corresponds to (or provides) a signal code, and the signal codes together in the sequence in which they are detected make up the signal code sequence.

Where the detectable signals provided by the hybridization probes are provided using fluorophores, the signal code for each decoding cycle may be the color of the fluorophore of the hybridization probe that hybridizes to the target nucleic acid sequence in that cycle. (As outlined above, in some embodiments, the hybridization probe may lack a detectable label, and thus the detectable signal may be the absence of a signal.) Accordingly, each nucleotide barcode sequence has a corresponding signal code sequence which may comprise a specific sequence of colors (which may include no color), which is distinct from the sequence of colors that makes up the signal code sequence of every other nucleotide barcode sequence. For example, the signal code sequence for a given target nucleic acid sequence may be R-G-B (wherein R represents a red fluorescent label, G represents a green fluorescent label, and B represents a blue fluorescent label). In some examples, in the first decoding cycle a red fluorescent label is used and a red signal is detected; in the second decoding cycle a green fluorescent label is used and a green signal is detected; and in the third decoding cycle a blue fluorescent label is used and a blue signal is detected. The individual signal codes (red, green and blue) are recorded, and in this manner the unique signal code sequence (R-G-B) which identifies the target nucleic acid sequence in question is assembled over time (in this case, over three decoding cycles). This system is capable of distinguishing between at least x^{y} different target nucleic acid sequences, wherein x is the number of different labels that are used and y is the number of individual signal codes in the signal code sequences.

In some embodiments, the detection method may involve the provision, for each target nucleic acid sequence, of a set of hybridization probes for decoding the signal code sequence. Each hybridization probe in a set comprises the same recognition sequence, such that the probes in the set hybridize to the same target nucleotide sequence in the target nucleic acid sequence, and a reporter probe binding site, which may be the same or different, and which is specific for a reporter probe, e.g. comprising a detectable label, or in some cases no label, which gives rise to a signal. The detectable signal from the reporter probe corresponds to an individual signal code. The hybridization probes are used in multiple cycles of decoding, performed sequentially in a pre-determined sequence, such that the signals detected from the hybridization probes which hybridize to the target nucleic acid sequence correspond to signal codes which make up the signal code sequence for that target nucleic acid sequence. To ensure that signals cannot be generated from the target nucleic acid sequences selected in step (b), e.g. to reduce signal crowding, the same set of antidote probes is used with the hybridization probes in each cycle of decoding. In some embodiments, hybridization probe-antidote probe complexes are formed between the hybridization probes for the selected target nucleic acid sequences and the antidote probes, such that signals which allow the selected target nucleic acid sequences to be identified are not generated. Signal crowding is thereby reduced.

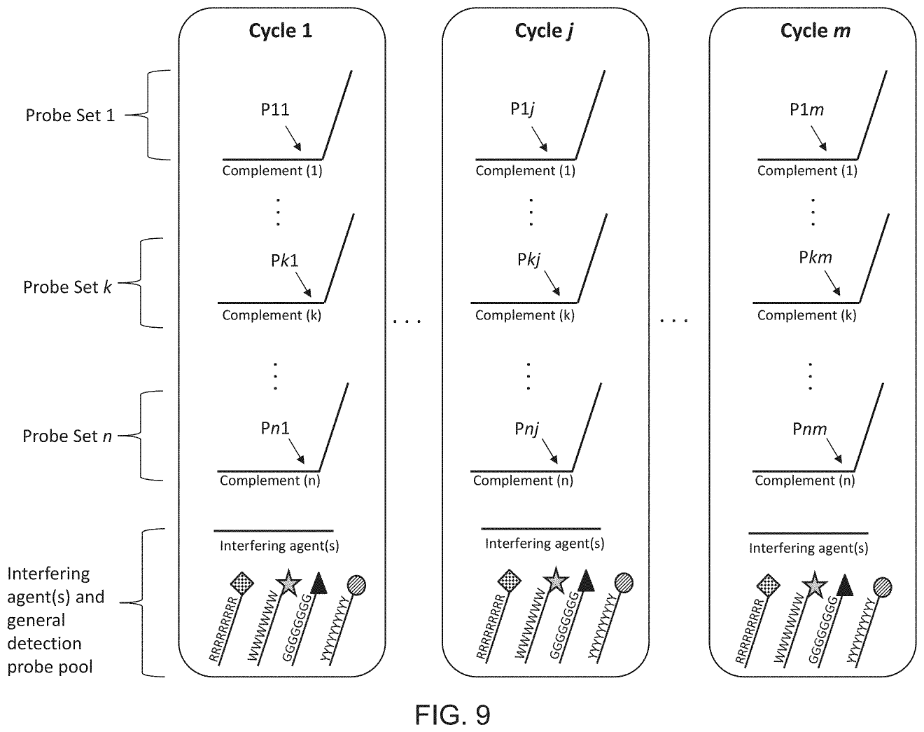

In some embodiments, target nucleic acid sequences T1, Tk, . . . , and Tn are analyzed using the n sets of hybridization probes in Cycle 1, . . . , Cycle j, . . . and Cycle m, wherein j, k, m, and n are integers, 2≤j≤m, and 2≤k≤n. In some embodiments, at least 5, 10, 20, 50, 100, 200, 500, 1,000, 2,000, 5,000, or 10,000 target nucleic acid sequences are analyzed using hybridization probes in 2, 5, 10, 20, 50, 100, or more cycles. In some embodiments as shown in FIG. 9, Hybridization Probe Set 1 comprises P11, . . . , P1j, . . . , and P1m, for target nucleic acid sequence T1, . . . , Hybridization Probe Set k comprises Pkl, Pkj, . . . , and Pkm, for target nucleic acid sequence Tk, . . . , and Hybridization Probe Set n comprises Pn1, . . . , Pnj, . . . , and Pnm, for target nucleic acid sequence Tn. For simplicity, target nucleic acid sequences are not depicted in FIG. 9. However, it will be understood that each probe set depicted in FIG. 9 corresponds to a target nucleic acid sequence, and a signal code sequence assigned to the target nucleic acid sequence can be decoded by sequentially hybridization of the probes in the set to said target nucleic acid sequence. In a given set, the probes can comprise the same recognition sequence that hybridizes to the target nucleic acid sequence (e.g., the complement of the target nucleic acid sequence, as depicted in FIG. 9).

For each cycle, the mixture of hybridization probes can be referred to as a hybridization probe library. The hybridization probes are used to decode signal code sequences for target nucleic acid sequences T1, . . . , Tk, . . . , Tn, by sequentially contacting the sample with Hybridization Probe Library P11, . . . , Pk1, . . . , and Pn1 and Ik (antidote for Pk1) in Cycle 1, . . . , with Probe Library P1j, . . Pkj, . . . , and Pnj and Ik (antidote for Pkj) in Cycle j, . . . , and with Probe Library P1m, . . . , Pkm, . . . , and Pnm and Ik (antidote for Pkm) in Cycle m (as shown in FIG. 9), where Tk is targeted by the interfering agent (antidote probe). The interfering agent (antidote) Ik for each cycle may be the same or different, and one or more of T1, . . . , Tk, . . . , and Tn may be targeted in each cycle, for example, wherein one or more antidote probes hybridize to the corresponding hybridization probe(s) and prevent them from hybridizing to the target nucleic acid sequences.

In some embodiments, target nucleic acid sequences T1, . . . , Tk, . . . , Tn comprise barcode sequences B1, . . . , Bk, . . . , Bn, respectively. In some embodiments, each hybridization probe in Probe Set 1, . . . , Probe Set k, . . . , and Probe Set n comprises recognition sequences R1, . . . , Rk, . . . , Rn (“complement” sequences shown in FIG. 9), that hybridize to barcode sequences B1, . . . , Bk, . . . , Bn, respectively. The interfering agents (antidotes) may hybridize to the target nucleic acid sequences or the hybridization probes. In some embodiments, one or more interfering agents (antidote probes) are provided to hybridize to recognition sequences R1, . . . , Rk, . . . , and/or Rn (e.g., by contacting the hybridizing probe library for each cycle with the antidote(s) before contacting the sample with the hybridizing probe library for the given cycle), thereby preventing the hybridizing probe(s) from hybridizing to the barcode sequence(s) of the target nucleic acid sequence(s). In some embodiments, any one or more of the target nucleic acid sequences can be a probe that directly or indirectly binds to a target nucleic acid analyte or a reporter nucleic acid for a target protein analyte or other non-nucleic acid analyte, or can be a product of the probe. The product can comprise one or more barcode sequences (e.g., corresponding to one or more analytes) and can be a hybridization product, a ligation product, an extension product (e.g., by a DNA or RNA polymerase), a replication product, a transcription/reverse transcription product, and/or an amplification product such as a rolling circle amplification (RCA) product. In particular embodiments, the target nucleic acid sequences comprise barcodes sequences in RCA products.

One exemplary workflow for a method disclosed herein is provided in FIG. 8A. For example, a tissue section can be analyzed with a highly multiplexed probe panel targeting multiple genes (e.g., RNAs) for detection using sequencing-by-hybridization (SBH). The sample can be contacted with a plurality of primary probes (e.g., padlock probes each complementary to the target nucleic acid sequences comprising one or more barcode sequences). The primary probes can be circularized (e.g., by ligation) and amplified by RCA. For the decoding of the barcode sequences present in the RCA products, pre-mixed pools of probes (e.g., hybridization probes, such as L-shaped or U-shaped hybridization probes) and sets of reporter probes can be contacted with the sample for sequentially decoding the plurality of target nucleic acid sequences. As shown in the figure, in some aspects, the method may first comprise performing reference SBH sequential decoding to identify highly abundant target nucleic acid sequences, which are selected for signal silencing by an interfering agent (“antidote”). The selected target nucleic acid sequences are also referred to as second target nucleic acid sequences. Optionally, a step of selecting the second target nucleic acid sequences is not included and the second target nucleic acid sequences are instead pre-determined (e.g., based on analytes known to be highly abundant in the tissue sample). Next, the workflow can include performing one or more “antidote” sequential decoding cycles. In some embodiments, each cycle of “antidote” decoding of first target nucleic acid sequences (sequences not selected for silencing by antidote) is performed using a pool of hybridization probes corresponding to the given cycle in order to generate assigned signal code sequences for the detected target nucleic acids. The pool of hybridization probes are provided and antidote probes are provided such that signals are generated for targets (e.g., first target nucleic acid sequences) except for the target nucleic acid sequences (e.g., second target nucleic acid sequences) selected for silencing. Multiple cycles of hybridization and/or detection of probes corresponding to target nucleic acids using sequential fluorescence hybridization and each cycle can comprise providing one or more antidote probes. In some embodiments, the same interfering agent or set of interfering agents is used in multiple “antidote” cycles or rounds. In one example, after sequential decoding of the first target nucleic acid sequences, one or more “dote” rounds is performed using “dote” hybridization probes (e.g., as shown in FIG. 8B) to detect one or more second (selected) target nucleic acid sequences. Alternatively, the one or more dote rounds can be performed before or in between rounds of “antidote” sequential decoding. As shown in FIG. 8A, the workflow can additionally include a step of hybridizing and detecting an anchor probe, wherein the anchor probe hybridizes to a common nucleic acid sequence comprised by or associated with a plurality of analytes in the sample. For example, a detectably labelled anchor probe can be hybridized in order to simultaneously detect all analytes (e.g., all RCPs) in the sample. The anchor probe hybridization and detection can be performed at any point in the workflow (e.g., before or in between any “antidote” or “dote” rounds). Finally, in some embodiments, the workflow can include overlaying data from the “antidote” and “dote” rounds in order to simultaneously visualize both lowly and highly abundant analytes (e.g., lowly and highly expressed genes) in the same sample.

### B. Interfering Agents (Antidotes)

The present disclosure provides various formats of interfering agents capable of preventing signals associated with one or more selected probes from being generated and/or detected, or capable of reducing the level of detected signal associated with one or more selected. In some embodiments, the interfering agents (also referred to interchangeably as antidote probes) disclosed herein eliminate or reduce signal generation and/or detection associated with selected probes by manipulation of the analyte/probe binding (e.g., hybridization) and/or the probe/detection agent binding (e.g., hybridization). In some embodiments, an interfering agents (antidote probe) disclosed herein eliminate or reduce signal generation and/or detection by quenching a detectable signal associated with a selected probe. In some embodiments, the interfering agents (antidote probes) disclosed herein eliminate or reduce signal generation and/or detection associated with selected probes by manipulation of hybridization, ligation, and/or amplification of selected circular or circularizable probe sets. The interfering agents disclosed herein may comprise any of a variety of entities that can hybridize to a nucleic acid (e.g., to a selected probe or a selected target nucleic acid sequence), typically by Watson-Crick base pairing, such as DNA, RNA, LNA, PNA, etc. In some embodiments, the interfering agents are interfering oligonucleotides (interfering oligos).

In some embodiments, the interfering agent (antidote probe) works by hybridizing to the selected hybridization probe (or second probe), or by hybridizing to the selected target hybridization sequence. These hybridization reactions may occur in any order. In some embodiments, the hybridization probes and the antidote probe are added to the sample simultaneously. In some embodiments, the hybridization probes are contacted with the at least one antidote probe first, such that the hybridization probe-antidote probe complex can form, before the sample is contacted with the hybridization and antidote probes. In some embodiments, the step (c) of contacting the sample with the hybridization probes of (a) and the at least one antidote probe of (b) may comprise contacting the hybridization probes of (a) with the at least one antidote probe of (b) to provide a hybridization probe/antidote probe mixture, and subsequently contacting the sample with the hybridization probe/antidote probe mixture. In some embodiments, antidote probes (e.g., for a target nucleic acid) can be provided each round in a plurality of sequential decoding rounds performed.

In some embodiments, there may be one or more steps of incubation to allow for the necessary hybridization reactions. In this regard, the hybridization probes may be incubated with the at least one antidote probe before they are added to the sample. Additionally or alternatively, the mixture of hybridization probes and hybridization probe-antidote probe complexes may be incubated with the target nucleic acid sequences present in the sample before signal detection occurs.

Contacting/incubation steps may be followed by one or more washing steps, e.g. to remove probes that have not hybridized.

In some embodiments, each antidote probe comprises a sequence which is complementary to a sequence within the selected hybridization probe or the corresponding target nucleic acid. In some embodiments, the antidote probe is complementary to at least a portion of the recognition sequence of the hybridization probe, or complementary to at least a portion of the target nucleic acid sequence which the hybridization probe recognizes. As such, the antidote functions to block or otherwise attenuate binding of the hybridization probe to the target nucleic acid molecule, thereby reducing or eliminating detection of a signal from the target nucleic acid molecule and consequently reducing signal crowding.

In some embodiments, the hybridization probe is indirectly labelled and comprises a reporter domain for hybridizing a detection probe/reporter probe, and the antidote probe may be complementary to at least a portion of the reporter domain. As such, the antidote functions to block or otherwise attenuate binding of the reporter probe (e.g., comprising a detectable label) to the hybridization molecule, thereby reducing or eliminating detection of a signal from the target nucleic acid molecule and consequently reducing signal crowding. In some embodiments, an antidote probe is complementary to the reporter domain of the hybridization probe, and the hybridization probe-antidote probe complex is able to bind to the target nucleic acid sequence, but the reporter probe will be unable to bind to the reporter domain of the hybridization probe, because it will be obstructed or blocked by the antidote probe. Thus, the hybridization probe-antidote probe complex is unable to provide a signal which allows the target nucleic acid sequence to be identified.

In some embodiments, the antidote probes are complementary to the reporter domain of the hybridization probe (and the hybridization probes are indirectly labelled using separate reporter probes), and the step of providing the one or more antidote probes occurs before the step of providing the set of reporter probes, such that the antidote probe can bind to the reporter domain of the hybridization probe before the cognate reporter probe. This will ensure that the hybridization probe-antidote probe complex can form correctly, so that a signal allowing the target nucleic acid sequence to be identified is not provided.

In some embodiments, the interfering agent (antidote probe) is capable of hybridizing to the hybridization probe to form a hybridization probe-antidote probe complex which is not capable of providing a signal which allows the selected target nucleic acid sequence to be detected. If the antidote probe is complementary to the recognition sequence of the hybridization probe, the complex formed between the antidote probe and the hybridization probe (the hybridization probe-antidote probe complex) will be unable to bind to the target nucleic acid sequence, because the recognition sequence of the hybridization probe will be obstructed, or blocked, by the antidote probe. In other embodiments, a complex is formed between the antidote probe and the target nucleic acid, and the complex is unable to bind to the hybridization probe.

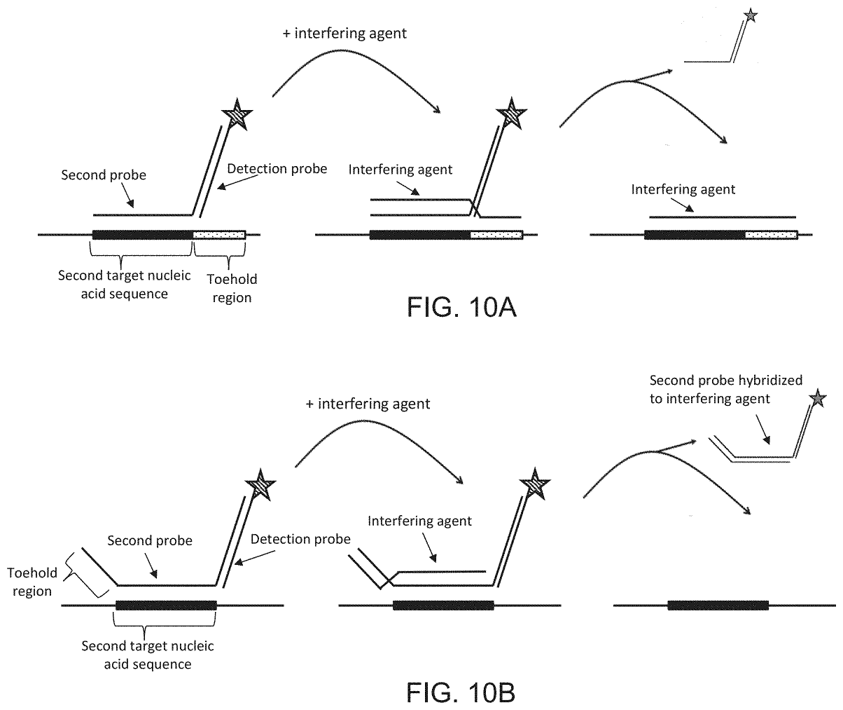

In some embodiments, the interfering agent (antidote probe) is capable of displacing the selected probe from its cognate target nucleic acid sequence, as shown in FIGS. 10A-10B. In some embodiments, the interfering agent comprises a sequence complementary to a toehold region that is a sequence adjacent to a selected target nucleic acid sequence, and a sequence complementary to the target nucleic acid sequence (as shown in FIG. 10A). In some embodiments, the toehold region is a single-stranded region in the target nucleic acid, and is available for hybridization by the interfering agent. In some embodiments, hybridization of the interfering agent to the toehold region can initiate a strand displacement reaction, wherein the interfering agent outcompetes the selected probe for hybridization to the selected target nucleic acid sequence, thereby displacing the selected probe. Although FIG. 10A depicts displacement of an L-shaped hybridization probe, it will be understood that the same or a similar interfering agent design could be used to displace any selected probe (e.g., a selected circular or circularizable probe or probe set). The displaced probe can be removed in a wash step. After displacement of the selected probe, the interfering agent can remain hybridized to the target nucleic acid sequence until it is optionally removed in a subsequent wash/stripping step.

In some embodiments, the selected probe can comprise a toehold region adjacent to the recognition sequence that hybridizes to the target nucleic acid sequence. For example, a toehold region can be located on an overhang of the probe, as shown in FIG. lOB, and the interfering agent can hybridize to the toehold region of the selected probe. The interfering agent can thus comprise a sequence complementary to the toehold region of the selected probe, and a sequence complementary to the recognition sequence of the selected probe, such that hybridization of the interfering agent to the toehold region initiates a strand displacement reaction that releases a hybridization complex comprising the selected probe hybridized to the interfering agent from the selected target nucleic acid sequence. The released hybridization complex can be removed in a wash step. Furthermore, although FIG. lOB depicts displacement of an L-shaped hybridization probe, it will be understood that the same or a similar strategy could be used to displace any selected probe (e.g., a selected circular or circularizable probe or probe set).

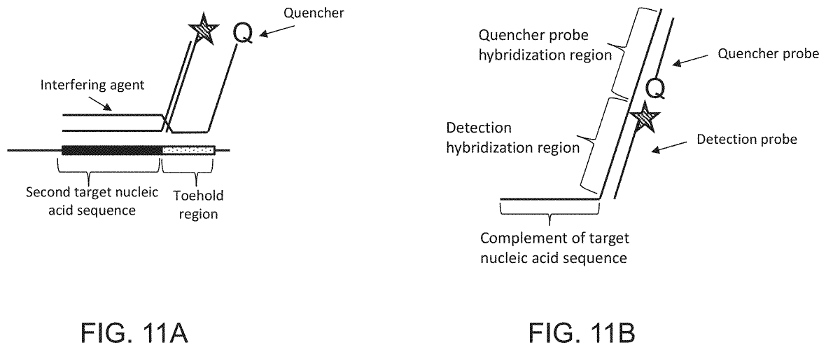

As discussed above, in some embodiments, an interfering agent can comprise a quencher moiety and can interfere with detection of a signal from selected probes (e.g., second probes) by quenching a detectable signal associated with said selected probes. In some embodiments, an interfering agent comprising a quencher moiety can also interfere with hybridization of a selected probe to its cognate target nucleic acid sequence, as shown in FIG. 11A. The inclusion of a quencher moiety can help further reduce or eliminate the signal associated with a selected probe. For example, in some embodiments, an interfering agent designed to displace a selected probe from a target nucleic acid may also comprise a quencher moiety, such that the quencher is brought into proximity with the detectable moiety (e.g., fluorescent moiety) of the selected probe during the displacement reaction, and can quench a signal of the detectable moiety if the interfering agent fails to completely displace the selected probe, as shown in FIG. 11A. Although FIG. 11A depicts an interfering agent comprising a quencher moiety hybridizing to a toehold region in the target nucleic acid (adjacent to the target nucleic acid sequence), it will be understood that the interfering agent comprising a quencher moiety could also be designed to hybridize to a toehold region within the probe, thereby initiating a strand displacement reaction (e.g., as shown in FIG. 10B).

In some aspects, an interfering agent can comprise a quencher moiety and can interfere with detection of a signal from selected probes, without interfering with hybridization of said probes to their cognate target nucleic acids. In some embodiments, a hybridization probe of a probe mixture for detection of a plurality of analytes comprises a recognition sequence capable of hybridizing to a particular target nucleic acid sequence (e.g., the complement of the target nucleic acids sequence), a detection hybridization region (reporter region), and a quencher probe hybridization region, as shown in FIG. 11B. In some embodiments, the quencher probe hybridization region corresponds to the recognition sequence (e.g., the quencher probe hybridization region is specific for the target nucleic acid sequence). In this way, interfering agents comprising quencher moieties (quencher probes) can be designed to hybridize to the quencher probe hybridization region of selected hybridization probes (e.g., hybridization probes corresponding to highly abundant analytes). Thus, the selected hybridization probes can be selectively targeted for quenching. As shown in FIG. 11B, in some embodiments, the quencher probe is hybridizes to the selected hybridization probe such that the quencher moiety is brought into proximity with the detectable label (e.g., fluorescent moiety) of the detection/reporter probe that is hybridized to the same selected hybridization probe, thereby specifically quenching the signal associated with the selected hybridization probe.

Suitable quenchers are known in the art. In some embodiments, the quencher is a non-fluorescent quencher. Non-fluorescent quenchers have been described, for example, in WO200608406 and in U.S. Pat. No. 7,019,129, the contents of which are herein incorporated by reference in their entirety. Commonly used non-fluorescent quenchers include DABCYL, TAIVIRA, BlackHole Quenchers™ (BHQ, e.g. BHQ, BHQ1, or BHQ2), Biosearch Technologies, Inc. (Novato, Cal.), Iowa Black™, Integrated DNA Tech., Inc. (Coralville, Iowa), BlackBerry™ Quencher 650 (BBQ-650), Berry & Assoc., (Dexter, Mich.).

In some embodiments, each of the at least one antidote probes is provided at a concentration that is the same as or higher than the concentration at which the hybridization probe to which it will bind is provided. Generally, the antidote probes are used in excess of the hybridization probes. In some embodiments, the concentration of the antidote probe is at least 1.5 times greater, such as at least 2, 3, 4, 5 or 10 times greater than the concentration of the corresponding hybridization probe. In some embodiments, the concentration of the antidote probe is any one of at least 15, 20, 25, 30, 35, 40, 50, 60, 70, 80, 90, 100, 200, 300, or more times greater than the concentration of the corresponding hybridization probe. In some embodiments, by using an increased concentration of antidote probe, relative to hybridization probe, the formation of the hybridization probe-antidote probe complex is favored. This ensures that a signal from the selected target nucleic acid sequence(s) is not detected or is strongly reduced.

In some embodiments, an antidote probe is provided at a final concentration (i.e., a final concentration in the probe mixture for contacting a sample) of between any of 0.5 uM and 1 uM, 0.75 uM and 1.5 uM, 1 uM and 2 uM, 1 uM and 5 uM, or 1 uM and 10 uM. In some embodiments, an antidote probe is provided at a final concentration of at least any one of 0.5 uM, 0.75 uM, 1 uM, 2 uM, 3 uM, 4 uM, or 5 uM. In some embodiments, an antidote probe is provided at a final concentration of no more than any one of 15 uM, 12 uM, 10 uM, 8 uM, 7.5 uM, 5 uM, 4 uM, 3 uM, 2 uM, or 1.5 uM.

### C. Signal Detection

In some embodiments, once the hybridization probes have hybridized to the target nucleic acid sequences, and the antidote probes have hybridized as necessary, the method comprises detecting a signal from each hybridization probe. This may involve detecting a signal from the detectable label of each hybridization probe which is directly or indirectly labelled with a detectable label. As noted above, signals are not detected from the hybridization probes for which antidote probes were provided, e.g. from the selected target nucleic acid sequences of step (b). Instead, signals are only detected from the hybridization probes for which cognate antidote probes were not provided. The presence of the antidote probes prevents signals being generated from the selected target nucleic acid sequences, and thus reduces signal crowding. This allows signals from the other, non-selected target nucleic acid sequences to be detected and resolved. Where a signal is absence of label, this may be distinguished from the case where signal is prevented by an antidote probe, since it is known which hybridization probes are selected for the antidote, and which and how other hybridization probes which are to be detected are labelled, or not. Accordingly, this can be accounted for in the analysis of the signals which are detected.

The signals may be detected by any suitable means known in the art for detecting the relevant detectable labels. In some embodiments, the signals may be detected by imaging the sample of target nucleic acid sequences. For example, if the detectable labels are fluorescent, the signals may be detected using fluorescence microscopy to determine the identity of the fluorescent label. It will be evident that other appropriate imaging techniques known in the art to identify signals from suitable detectable moieties may be used in the present methods to detect a signal from the label of a hybridization probe.

In some embodiments, the step of detecting signal from the labels of the hybridization probes which have hybridized to their respective target sequences may further comprise a step of removing unhybridized probes, prior to detecting the signals. The removal of the unhybridized probes may improve the strength of the signal that is detected, or the signal to noise ratio. This removal step may be carried out by washing the target nucleic acid sequences with an appropriate wash buffer. The step of washing the may be repeated multiple times, e.g. 2, 3, 4, 5 or more times, as is necessary.

In some embodiments, the signals that are detected from the hybridization probes which have hybridized to their target sequences allow for the target nucleic acid sequences which are not targeted by antidote probe(s) (e.g., where hybridization probes are contacted with antidote probes before contacting with the sample) to be identified based on the signals detected, thereby detecting the target nucleic acid sequences within the sample.

In some embodiments, each hybridization probe is capable of giving rise to signal by being detected, either directly or indirectly. As noted above, this may be the presence or absence of signal. Different hybridization probes may be detected, or distinguished from one another, by different labels, or by absence of a detectable label. In some embodiments, each hybridization probe may be directly or indirectly labelled with a detectable label which gives rise to a signal which may be recorded and/or assigned (e.g., serially) a signal code. In some embodiments, each hybridization probe is capable of hybridizing to a different target nucleic acid sequence (e.g., barcode sequence corresponding to a target analyte) and providing a signal. In some embodiments, a signal may include the signal detectable from the detectable label, and different detectable labels may provide different signals which may be distinguished, e.g. by color. In some embodiments, absence of signal may also be recorded and/or assigned a signal code. In some embodiments, in a plurality of hybridization probes, one or more of the probes may be lacking a detectable label, and thus the absence of a signal may be recorded and analyzed, for example, by assigning a signal code to the absence of signal (also known as a “dark” cycle for the one or more of the probes and the corresponding analyte(s)). In some embodiments, when there is a single cycle of detection to detect the signals from the hybridization probes, the plurality of hybridization probes may comprise molecules of one hybridization probe which is not labelled, and the remainder of the probes may comprise detectable labels which can be distinguished from one another. In some embodiments, a combinatorial, e.g. sequential, labelling scheme is used (e.g., multiple cycles of sequential signal detection), and the plurality of hybridization probes for different analytes (or different barcode sequences corresponding to the same or different analytes) used in a given cycle need not all be distinguishable from one another in terms of the signal (e.g., may comprise the same detectable label, such as the same color of fluorophore), as it is the combination (e.g., sequence or order) of signals which identifies the target nucleic acid sequence, not a single signal.

The detectable label may be any detectable moiety and may be directly or indirectly linked to the hybridization probe. The hybridization probe may thus be considered to be directly or indirectly signal-giving. In some embodiments, the detectable label is incorporated into the hybridization probe. For example, the detectable label may be linked directly (e.g., covalently) or via a linker (e.g., a chemical or nucleic acid linker) to the target nucleic acid recognition sequence of the hybridization probe.

In some embodiments, the hybridization probe (e.g., bound to the target nucleic acid) may provide a signal indirectly, e.g., via one or more further components (e.g., detectably labeled probes that bind the hybridization probe) to generate a signal. For instance, the hybridization probe may comprise a domain which is capable of binding a species that comprises a detectable label. In some embodiments, the hybridization probe comprises a detection hybridization region (also referred to as a reporter domain) which is not complementary to, and does not bind to, the target nucleic acid sequence but which comprises a binding site for a detection probe (also referred to as a reporter probe) which comprises a detectable label. More particularly, the detection hybridization region/reporter domain of the hybridization probe may comprise a binding site in the form of a nucleotide sequence comprising a region or domain to which a complementary detection/reporter probe may hybridize. In some embodiments, the nucleotide sequence of the detection/reporter domain is not complementary to and does not hybridize to the target nucleic acid sequence.

In some embodiments, the detection/reporter domain may be in the form of an overhang region of the hybridization probe, which is not complementary to the target nucleic acid sequence, but which comprises a binding site that is complementary to the sequence of a detection/reporter probe. In some embodiments, the detection/reporter probe comprises a cognate sequence which is complementary to that of the binding site in the reporter domain, and a detectable label.

In some embodiments, a method disclosed herein comprises providing a plurality of hybridization probes each specific for a target nucleic acid and a set of detection probes/reporter probes, cognate for the hybridization probes. The detection/reporter probes may be used separately from the hybridization probes and that they do not necessarily need to be provided together or at the same time. For instance, the detection/reporter probes (as well as the interfering agents such as antidote probes) may be contacted with the sample at a separate time, or in separate step, to contacting with the hybridization probes. For example, the sample may be contacted with the detection/reporter probes after contacting with the hybridization probes and the antidote probes, for example during the detecting step. In some embodiments, a detection/reporter probe and a hybridization probe are cognate to each other in that the detection/reporter probe corresponds to and is designed to bind to the hybridization probe (e.g., via a reporter domain on the hybridization probe) .

In some embodiments, a detection/reporter probe (e.g., a fluorescently labelled detection oligo) herein comprises a sequence which is complementary to that of a reporter domain (the detection hybridization region or reporter probe binding site) in a hybridization probe. In some embodiments, each detection/reporter probe comprises a detectable label. In some embodiments, a plurality of different sets of detection/reporter probes are provided, each set with a type of detectable label. The detectable label for each set of detection/reporter probes may be different, for example, the detectable label for each set can be a different fluorophore detectable in a separate fluorescence channel of a microscope. The plurality of hybridization probes and the sets of reporter probes may be provided simultaneously or sequentially. In an embodiment, a mixture of hybridization probes and detection/reporter probes may be prepared and added to or contacted with the sample. Further, in some embodiments, the reporter probes may be pre-hybridized to the hybridization probes. In other embodiments, the detection/reporter probes are hybridized to the hybridization probes after they have hybridized to their target sequence(s), or after the hybridization probes have been allowed to hybridize to their target sequence(s), and the antidote probes have been allowed to hybridize to the hybridization probes or the target sequence(s).

Detectable labels that may be used according to the methods herein, either in hybridization probes, or in detection/reporter probes, include any moiety capable of providing a signal that can be detected, for example fluorescent molecules (e.g. fluorescent proteins or organic fluorophores), colorimetric moieties (e.g. colored molecules or nanoparticles), particles, for example gold or silver particles, quantum dots, radioisotopes, chemiluminescent molecules, and the like. Any detectable moiety may be used as the detectable label. In particular, any spectrophotometrically or optically detectable label may be used. In some embodiments, the detectable label may be optically detectable. The detectable label may be distinguishable by color, but any other parameter may be used e.g. size or intensity.

In an embodiment, the hybridization probe or the reporter probe comprises a fluorescent label. This may be a fluorescent molecule, e.g. a fluorophore. Fluorescent molecules that may be used to label nucleotides are well known in the art. Exemplary fluorophores include ATTO dyes (such as ATTO 425, ATTO 550, ATTO 647(N), ATTO 655), cyanine dyes (e.g., Cy3, Cy5, Cy7), and Alexa Fluor dyes (such as AF 488, AF555, AF 647, AF 750), though any suitable fluorophores may be used. Fluorophores have been identified with excitation and emission spectra ranging from UV to near IR wavelengths. Thus, the fluorophore may have an excitation and/or emission wavelength in the UV, visible or IR spectral range. In some instances, the fluorophore is a green fluorescent protein, a blue fluorescent protein, a yellow fluorescent protein, an orange fluorescent protein, a red fluorescent protein, a far-red fluorescent protein, or a near-IR fluorescent protein, or any combinations thereof. The fluorophore may be a peptide, small organic compound, synthetic oligomer or synthetic polymer. In some embodiments, the fluorophore is a small organic compound.

In some embodiments, a reporter probe (also referred to as detection probe, e.g., detection oligo) may comprise no detectable label. In this case, the signal that is reported is the absence of any detectable label, which is distinguishable from any number of distinct positively detectable labels.

### V. Kits

In some aspects, provided herein are kits for analyzing an analyte in a biological sample according to any of the methods described herein. In some embodiments, provided herein is a kit comprising one or more of the probes disclosed herein, such as any of the circular probes or circularizable probes or probe sets, hybridization probes, interfering agents (antidote probes), dote probes, and/or detection/reporter probes described herein. In some embodiments, the kit comprises anchor probes. In some embodiments, a set of nucleic acid probes are designed and provided for each target and the kit may comprise a plurality of sets of nucleic acid probes for a plurality of targets. In some embodiments, the kit comprises pre-mixed pools or libraries of probes (e.g., hybridization probes, such as L-shaped or U-shaped hybridization probes) for sequentially decoding a plurality of target nucleic acid sequences.

In some aspects, provided herein is a kit for use in the above-mentioned method of detection, the kit comprising: (a) a plurality of hybridization probes comprising different hybridization probes each specific for a different target nucleic acid sequence (e.g., reporter sequence or barcode associated with a given analyte), wherein each hybridization probe has a recognition sequence complementary to a sequence within its target nucleic acid sequence, and is capable of giving rise to a signal by means of which it can be detected; and (b) at least one antidote probe for at least one selected hybridization probe specific for a selected target nucleic acid sequence. In some embodiments, each antidote probe comprises a sequence complementary to a sequence within the selected hybridization probe and is capable of hybridizing to it to form a hybridization probe-antidote probe complex which is not capable of providing a signal which allows the selected target nucleic acid sequence to be detected. In some embodiments, each antidote probe comprises a sequence complementary to a sequence within the selected target nucleic acid sequence and is capable of hybridizing to it to form a selected target nucleic acid sequence-antidote probe complex which is not capable of providing a signal which allows the selected target nucleic acid sequence to be detected, for example, because the antidote probe prevents or reduces hybridization of the selected hybridization probe to the selected target nucleic acid sequence.

The disclosures above relating to the structures of the hybridization probes and the antidote probes apply equally in relation to the hybridization probes and antidote probes which make up the kit. In some embodiments, the methods disclosed herein comprise providing a plurality of hybridization probes comprising probes specific for different target nucleic acid sequences (e.g., different reporter or barcode sequences associated with a given analyte) and at least one antidote probe disclosed herein. The plurality of hybridization probes may be a mixture of hybridization probes which are complementary to the target nucleic acid sequences which are to be detected. Thus, a composition may be provided comprising such a plurality or mixture of multiple hybridization probes. In some embodiments, the plurality of hybridization probes comprises a different hybridization probe for each target nucleic acid sequence (e.g., barcode sequence) to be detected.

A composition or mixture comprising multiple hybridization probes, or alternatively put, a plurality of hybridization probes, may be considered to be a general or universal hybridization probe mix. In many multiplex methods, it is advantageous to have a consistent hybridization probe mix which can be used to detect a range of target nucleic acid sequences in a number of different reactions, rather than to re-formulate a separate hybridization probe mix for each reaction. For example, as noted above, it may be desired to detect target nucleic acid sequences in order to analyze the gene expression or protein composition of a particular cell sample. For example, if multiple cell or tissue samples are to be analyzed in order to compare gene expression across different cell types or tissue samples or sections, it may be easier, cheaper, and quicker to use a single hybridization probe mixture for analyzing all of the cell samples, than to design a separate hybridization probe mix for each sample.

In some embodiments, the number of antidote probes is less than the number of hybridization probes, e.g., in cases where one antidote probe is provided for each target nucleic acid sequence for which a signal is to be suppressed or silenced. In some embodiments, the kit is arranged such that not every hybridization probe has a corresponding antidote probe. In some embodiments, the number of antidote probes in the kit may be less than 50%, such as less than 40%, 30%, 25%, 20%, 15%, 10% or 5% of the number of hybridization probes in the kit.

In some embodiments, the kit comprises n sets of probes:

Probe Set 1 comprises P11, . . . , P1j, . . . , and P1m, for target nucleic acid sequence T1,

Probe Set k comprises Pk1, . . . , Pkj, . . . , and Pkm, for target nucleic acid sequence Tk,

Probe Set n comprises Pn1, . . . , Pnj, . . . , and Pnm, for target nucleic acid sequence Tn,

wherein j, k, m, and n are integers, 2≤j≤m, and 2≤k≤n, and the n sets of probes are used to decode signal code sequences for target nucleic acid sequences T1, . . . , Tk, . . . , Tn, in m cycles. In some embodiments, each probe is detectable by a fluorescently labelled reporter probe, and the fluorescent signals for different probes in each probe set or each probe library can be of the same or different colors. In some embodiments, n (the number of target nucleic acid sequences to be detected) is at least 5, 10, 20, 50, 100, 200, 500, 1,000, 2,000, 5,000, or 10,000, or greater than 10,000. In some embodiments, m (the number of cycles) is at least 2, 3, 4, 5, 6, 7, 8, 9, 10, 11, 12, 13, 14, 15, 16, 17, 18, 19, 20, or greater than 20. In some embodiments, the sample is contacted with Probe Library P11, . . . , Pk1, . . . , and Pn1 in Cycle 1, with Probe Library P1j, . . . , Pkj, . . . , and Pnj in Cycle j, and Probe Library P1m, . . . , Pkm, . . . , and Pnm in Cycle m.

In some embodiments, in one or more of Cycle 1 through Cycle m, the sample is contacted with an interfering agent (e.g., an interfering oligo) that hybridizes to a target nucleic acid sequence or the corresponding probe(s), and the interfering agent (e.g., an interfering oligo) is not detectably (e.g., fluorescently) labelled. In some embodiments, the kit further comprises interfering agents (e.g., interfering oligos) that hybridizes to all of the probes in any one or more of Probe Set 1 to Probe Set n. For example, when target nucleic acid sequence Tk is an abundant or highly expressed in the sample, an interfering agent (e.g., an interfering oligo) Ik that hybridizes to Tk or the corresponding probes Pk1, . . . , Pkj, . . . , and Pkm may be included in the kit. In some examples, interfering agent (e.g., interfering oligo) Ik hybridizes to all of probes Pk1, . . . , Pkj, . . . , and Pkm and prevents these probes from hybridizing to Tk and providing detectable signals indicative of Tk in the sample. For instance, the sample can be contacted with Probe Library P11, . . . , Pk1, . . . , and Pn1 and Ik (antidote for Pk1) in Cycle 1, with Probe Library P1j, . . . , Pkj, . . . , and Pnj and Ik (antidote for Pkj) in Cycle j, and with Probe Library P1m, . . . , Pkm, . . . , and Pnm and Ik (antidote for Pkm) in Cycle m. In some embodiments, the kit comprises hybridization probes and antidote probes for each cycle in a pre-mix which is then contacted with the sample. In some embodiments, a kit disclosed herein comprises any one or more of m compositions: Composition No. 1 comprising P11, . . . , Pk1, . . . , and Pn1 and Ik (antidote for Pk1), . . . , Composition No. j comprising P1j, . . . , Pkj, . . . , and Pnj and Ik (antidote for Pkj), . . . , and Composition No. m comprising P1m, . . . , Pkm, . . . , and Pnm and Ik (antidote for Pkm). Exemplary compositions (corresponding to decoding cycles) are depicted in FIG. 9.

In some embodiments, the kit further comprises a probe set for one or more “dote” cycles before Cycle 1, in between any of Cycles 1 to m, or after Cycle m. For example, the probe set for a dote cycle may include one or more “dote” probes from P11, . . . , P1j, . . . , and P1m, . . . , Pk1, . . . , Pkj, . . . , and Pkm, and Pn1, . . . , Pnj, . . . , and Pnm.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents for performing a nuclease digest described herein, such as one or more restriction endonuclease enzymes and buffers for restriction digest reactions. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits optionally contain other components, for example nucleic acid primers, enzymes and reagents, buffers, nucleotides, modified nucleotides, reagents for additional assays.

### VI. Applications

The methods disclosed herein are methods for detecting multiple target nucleic acid sequences in a sample. These methods may be used in a variety of different applications, and thus the identity of the sample and of the target nucleic acid sequences may be varied. Any means of determining the presence of the target nucleic acid sequences (e.g., if they are present or not) or any form of measurement of the target nucleic acid sequences may be employed. A method disclosed herein may comprise determining, measuring, assessing and/or assaying the presence or absence or amount or location of the target nucleic acid sequences in any way.

In some embodiments, a method disclosed herein may comprise using a sequential decoding scheme for localized detection of target nucleic acid sequences in a sample. In some embodiments, the antidote probe method may be performed with only a single round of detection of hybridized hybridization probes, e.g., a single step of detecting the signals. In some embodiments, in a localized detection, the signals giving rise to the detection of the target nucleic acid sequences are localized to those sequences. In turn, the target nucleic acid sequences are localized in the sample, that is they are present at, and remain at, a given or specific location in the sample. The target nucleic acid sequences may therefore be detected in or at their locations in the sample. In some embodiments, the spatial position (or localization) of the target nucleic acid sequences within the sample may be determined (or “detected”). This means, for example, that the target nucleic acid sequences may be localized to, or within, a cell or tissue in which they are expressed, or to a position within a cell or tissue sample in which they are present. A target nucleic acid sequence which is not itself the target analyte of an assay, but which is generated therefrom, or used or generated as a reporter therefor may be localized to the target analyte, and hence in the sample, by virtue of being bound to or otherwise associated with the analyte. Thus “localized detection” may include determining, measuring, assessing or assaying the presence or amount and location, or absence, of the target nucleic acid sequences in any way.

More particularly, the methods may be used for the in situ detection of target nucleic acid sequences, or of a target analyte for which the target nucleic acid sequence is a reporter. In a particular embodiment, the methods may be used for the localized, particularly in situ, detection of mRNA sequences. More particularly, the methods may be used for the localized, particularly in situ, detection of mRNA sequences in a sample of cells.

In some embodiments, an in situ assay comprises the detection of target nucleic acid sequences, or target analytes, in their native contexts, e.g. in the cell or tissue in which they normally occur. Thus, this may refer to the natural or native localization of target nucleic acid sequences or target analytes. In other words, the target nucleic acid sequences may be detected where, or as, they or the target analytes in respect of which they are to be detected, occur in their native environment or situation. Thus, the target nucleic acid sequences or analytes are not moved from their normal location, e.g. are not isolated or purified in any way, or transferred to another location or medium etc. In some embodiments, an in situ assay comprises the detection of the target nucleic acid sequences or analytes as they occur within a cell or within a cell or tissue sample, e.g. their native localization within the cell or tissue and/or within their normal or native cellular environment. In particular, in situ detection includes detecting the target nucleic acid sequences within a tissue sample, and particularly a tissue section. In other embodiments the methods can be carried out on a sample of isolated cells, such that the cells themselves are not in situ. In some embodiments, the in situ assay is a fluorescence in situ hybridization (FISH) assay wherein the interfering probes are added to one or more cycles to interfere with binding of one or more of the probes to a target nucleic acid sequence or binding of the fluorescently labelled reporter probe to the one or more probes that bind to a target nucleic acid sequence.

In some embodiments, a method disclosed herein comprises multiple sequential decoding cycles, wherein the signal code sequence for each target nucleic acid sequence is determined by detecting signals from individual hybridization probes across multiple cycles. Indeed, as also noted above, this also applies to any sequential decoding scheme, including in the context of the “antidote probe” method. It will therefore be understood, that in order for the signal code sequences for each target nucleic acid sequence to be built up, the target nucleic acid sequences must be fixed in position, or immobilized. If the target nucleic acid sequences were not each located at a single site or position (e.g., immobilized) in the sample, it would not be possible to identify a sequential set of signals which were detected from the same target nucleic acid sequence, and thus the signal code sequences could not be correctly determined. In some embodiments, this immobilization may occur by virtue of the target nucleic acid sequences being present in situ in a sample, or being bound to or associated with a target analyte which is present in situ. In other embodiments, this may be done by immobilizing the target nucleic acid sequences in situ, for example, the target nucleic acid sequences may be immobilized, or fixed, as they occur in the sample, e.g. in cells. For example, a tissue sample may be fixed or immobilized, or cells may be taken from a sample, which may be tissue or body fluid sample, or indeed a culture or any sample containing cells, and the cells may be fixed or immobilized onto a solid surface. In such a situation, whilst the cells may no longer be in a native in situ context, the target analyte/nucleic acid sequence may remain in an in situ context within the cell. In still other embodiments, the target nucleic acid sequences, or their corresponding target analytes (e.g. the analytes to which they bind or become bound or associated etc.) may be removed from their native in situ context and immobilized on a solid surface. In this way, the target nucleic acid sequence or target analyte may be localized at a particular identifiable site or location, and may remain there during the performance of the method such that, in particular, the location does not change, and remains the same, from cycle to cycle.

Accordingly, a method disclosed herein is not necessarily limited to localized detection in situ; the target nucleic acid sequences may alternatively be localized by being immobilized on a solid support not in the context of their original or native location. In this context, the target nucleic acid sequences are isolated from their original environment, and thus it will be understood that information about the location of the target nucleic acid sequences within that environment will not be available.

It will be seen from the above that where the method does not require sequential cycles, namely in some embodiments of the antidote method, the detection of the target nucleic acid sequences need not be localized. Further, both methods include embodiments which are not in situ, in the sense that the target nucleic acid sequences, or their corresponding or respective target analytes, are not present (e.g. not fixed) in their native contexts. This may include embodiments in which target nucleic acid sequences are immobilized, directly or indirectly, e.g. on a solid support. In still other embodiments of the “antidote probe” method, the method can be carried out in solution or in suspension. In particular the target nucleic acid sequences can be in solution. Thus, for example, the method can be performed on a sample comprising isolated target nucleic acid sequences.

The target nucleic acid sequences are present within a sample. The sample may be any sample which contains any amount of target nucleic acid sequences which are to be detected, from any source or of any origin. A sample may thus be any clinical or non-clinical sample, and may be any biological, clinical or environmental sample in which the target nucleic acid sequences may occur. All biological and clinical samples are included, e.g. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared for use in the methods of the present invention, or they may be prior-treated in any convenient way e.g. for storage.

As noted above, in one embodiment, the target nucleic acid sequences may be detected in situ, as they naturally occur in the sample. In such an embodiment the target nucleic acid sequences may be present in a sample at a fixed, detectable or visualisable position in the sample. The sample will thus be any sample which reflects the normal or native (“in situ”) localization of the target nucleic acid sequences, e.g. any sample in which they normally or natively occur. Such a sample will advantageously be a cell or tissue sample. Particularly preferred are samples such as cultured or harvested or biopsied cell or tissue samples in which the target nucleic acid sequences may be detected to reveal the localization of the target nucleic acid sequences relative to other features of the sample. Thus, the in situ context may be the context of a cell. In another embodiment the in situ context may be the in the context of a tissue which contains the cell, etc. Accordingly, in some embodiments, the sample may be a cell or tissue sample, in particular a human tissue sample. In some embodiments, the sample may be a cancer tissue sample.

As well as cell or tissue preparations, such samples may also include, for example, dehydrated or fixed biological fluids, and nuclear material such as chromosome/chromatin preparations, e.g. on microscope slides. The samples may be freshly prepared or they may be prior-treated in any convenient way such as by fixation or freezing. Accordingly, fresh, frozen or fixed cells or tissues may be used, e.g. FFPE tissue (Formalin Fixed Paraffin Embedded).

Thus, representative samples may include any material which may contain target nucleic acid sequences to be detected, including for example foods and allied products, clinical and environmental samples, etc. The sample may be a biological sample, which may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue-green algae, fungi, bacteria, protozoa etc. Representative samples thus include clinical samples, e.g. whole blood and blood-derived products such as plasma, serum and buffy coat, blood cells, other circulating cells (e.g. circulating tumor cells), urine, faeces, cerebrospinal fluid or any other body fluids (e.g. respiratory secretions, saliva, milk, etc.), tissues, biopsies, as well as other samples such as cell cultures, cell suspensions, conditioned media or other samples of cell culture constituents, etc. The sample may be pre-treated in any convenient or desired way to prepare for use in the methods of the present invention, for example by cell lysis or purification, fixing of cells, immobilization or isolation of the target nucleic acid sequences, etc.

Similarly, the target nucleic acid sequences within the sample may be any target nucleic acid sequences which it is desired to detect. In some embodiments, the target nucleic acid sequence may be target analyte nucleic acid sequences. The target analyte nucleic acid sequences may be any nucleic acid sequences, including DNA, RNA, or a mixture thereof. Moreover, the target analyte nucleic acid sequences may be any form of nucleic acid, such as mRNA, cDNA, etc. As noted above, in a particularly preferred embodiment, the target nucleic acid sequences are mRNA sequences.

The target nucleic acid sequences may be nucleic acid sequences generated from target analyte nucleic acid sequences, such as amplicons or complementary copies of target analyte nucleic acid sequences. In some embodiments, the RNA sequences present in the sample may be reverse transcribed into cDNA sequences, for example by contacting the sample with a reverse transcriptase enzyme and appropriate primers. Such enzymes and primers are well known in the art, and any suitable enzymes and primers may be employed. In such an embodiment, the cDNA sequences produced by the reverse transcription reaction can then be considered as the target nucleic acid sequences to be detected.

Furthermore, the target nucleic acid sequences may be nucleic acid sequences which are generated as reporters for other target analytes. In such an embodiment, target nucleic acid sequences may be provided to, e.g. added to or generated in a sample (e.g. they may be nucleic acid sequences that were not present in the original sample). A target nucleic acid sequence may be provided in the sample as a tag or reporter for a target analyte, for example by one or more molecules that interact with, e.g. bind to, target analytes. The detection of the added or generated target nucleic acid sequences is thus indicative of the presence of the alternative target analytes in a sample.

In such a context the target analyte may be any target molecule, including nucleic acid molecules, or analytes other than nucleic acid molecules, such as a proteins, peptides, carbohydrates etc. Various methods based upon detecting such a reporter target nucleic acid sequence, which is indicative of an underlying target analyte, are well described in the art, including, for example, immunoRCA and immunoPCR as noted above, and assays using padlock probes or proximity probes. The use of proximity probes comprising analyte-binding domains and nucleic acid domains which interact upon binding of the probes to a target analyte is widely known and described in the literature. In the context of proximity probes, a target nucleic acid sequence may be generated by extension or ligation of nucleic acid domains of proximity probes, or of additional nucleic acid molecules (e.g. oligonucleotides) which hybridize to the nucleic acid domain of a proximity probe. In the context of a padlock probe, a target nucleic acid sequence may be generated as the RCP of that probe, or indeed simply a result of ligation of the probe. In the context of immunoPCR or immunoRCA, a PCR or RCA product may be generated to provide a target nucleic acid sequence. Still further, a target nucleic acid sequence may be added to the sample as the nucleic acid domain of an analyte-binding probe.

For example, a protein or other analyte in a sample may be detected by an antibody or other analyte-specific binding partner, which is provided with an oligonucleotide, and that oligonucleotide may be considered to be a target nucleic acid sequence. In this case, the hybridization probes may be designed to hybridize to that oligonucleotide, such that the oligonucleotide can be detected, and therefore can indicate the presence of the antibody, and hence the analyte. Similarly, an oligonucleotide sequence may be generated as part of an analyte detection assay, e.g. an extension or ligation product may be generated as the result of a proximity assay, and this oligonucleotide may be considered to be a target nucleic acid sequence.

In some embodiments, a circularizable probe or probe set specific for each target nucleic acid sequence is circularized upon hybridization to the target nucleic acid sequence and is amplified by rolling circle amplification (RCA) to produce a rolling circle product (RCP). As noted above, the target nucleic acid sequences to which the circularizable probes are hybridized may be any nucleic acid sequences which are desired to be detected. The RCA reaction is used to amplify the signal which is generated from the target nucleic acid sequence, in order to improve the signal to noise ratio and therefore increase the utility of the detection method. In the context of the “antidote probe” detection method, in order to obtain the same advantages associated with signal amplification, in some embodiments, the target nucleic acid sequences may be rolling circle amplification products (RCPs) generated from circularizable probes.

The circularizable probes or probe sets from which the RCPs are generated may be as described above. The circularizable probes or probe sets can be circularized (e.g., by templated or non-templated ligation) before the RCA reactions occur. In particular, the circularizable probes may be padlock probes. As outlined above, the padlock probes may each comprise a barcode sequence, wherein each padlock probe comprises a different barcode sequence specific for a different target nucleic acid sequence.

In some embodiments, the padlock probes may be specific for target nucleic acid analytes present in the sample. In a particular embodiment, the method may be used for the detection of mRNA sequences, and thus the padlock probes may be specific for mRNA sequences present in the sample. More particularly, the sample may be a sample of cells, and the mRNA sequences may be detected in situ. Accordingly, the padlock probes may hybridize to and circularise on the mRNA sequences present in the cells in the sample. When the padlock probes are amplified by RCA, the resulting RCPs will each comprise multiple complementary copies of the barcode sequence from the relevant padlock probe. The barcode sequences will allow the RCPs (the target nucleic acid sequences) to be detected using the decoding methods outlined above, and therefore will in turn allow the mRNA sequences to be indirectly detected.

The present detection methods reduce signal crowding by reducing the number of signals which are generated and detected at any one time. As set out above, this may be done via the use of antidote probes, and it is possible to vary the extent to which these strategies are employed (e.g. to vary the number of targets and/or hybridization probes that are targeted by antidote probes), depending on the degree of signal crowding which is experienced.

In some embodiments, any one or more target nucleic acid sequences may be selected and antidote probes are provided. In some embodiments, an antidote probe may hybridize to a target analyte (e.g., a target nucleic acid analyte) or a reporter nucleotide associated with a target analyte (e.g., a protein or other non-nucleic acid analyte). In some embodiments, an antidote probe may hybridize to a probe (e.g., a circularisable probe) or probe set (e.g., a SNAIL probe set) that hybridizes to a target analyte, or an amplification product (e.g., RCP) of the probe or probe set. In some embodiments, an antidote probe may hybridize to a hybridization probe that hybridizes to an amplification product (e.g., RCP) of a probe (e.g., a circularisable probe) or probe set (e.g., a SNAIL probe set) that corresponds to a target analyte, or the antidote probe may hybridize to a detectably labelled oligo that hybridizes to the hybridization probe. In some embodiments, in order to reduce the signal crowding to the largest possible extent, it may be desirable to target the signal crowding reduction strategies against the specific target nucleic acid sequences which are significantly responsible for causing the signal crowding problem. Accordingly, in some embodiments, the target nucleic acid sequences which are selected (to be targeted by using antidote probes for the target sequences or hybridization probes) are target nucleic acid sequences which are present in the sample in an increased amount relative to other target nucleic acid sequences in the sample.

For example, if the detection methods are to be used to detect multiple target mRNA sequences in order to assess gene expression in a particular cell or tissue sample, the target nucleic acid sequences which are selected (for which antidote probes are selected and/or provided) may be target mRNA sequences corresponding to a gene or genes which is expressed in an increased amount relative to other genes in the sample.

The selection of target nucleic acid sequences which are present in the sample in an increased amount relative to other target nucleic acid sequences may be informed by prior knowledge of the sample in question. In the example provided above of a gene expression analysis, the skilled person may be aware of genes which are likely to be highly expressed within the sample in question, and may select target nucleic acid sequences accordingly. That is to say that the skilled person may be able to use the common general knowledge in the field to select appropriate target nucleic acid sequences.

Alternatively, the selection may be based on the results of previous experiments. In this regard, in some embodiments, the detection methods may comprise preceding steps of identifying target nucleic acid sequences which cause signal crowding. For example, one or more target nucleic acid sequences may have been previously detected without using any interfering agents and observed to cause signal crowding.

In the context of the “antidote probe” detection method, the method may comprise preceding steps of detecting target nucleic acid sequences in the sample using the plurality of hybridization probes of step (a) and determining the presence in the sample of target nucleic acid sequences which give rise to signals which crowd out signals from other target nucleic acid sequences in the sample, wherein those target sequences are selected for step (b).

### VII. Terminology

Specific terminology is used throughout this disclosure to explain various aspects of the apparatus, systems, methods, and compositions that are described.

Having described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Adaptor, Adapter, and Tag

An “adaptor,” an “adapter,” and a “tag” are terms that are used interchangeably in this disclosure, and refer to species that can be coupled to a polynucleotide sequence (in a process referred to as “tagging”) using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences.

### (vi) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

(vii) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(viii) Primer Extension

A “primer extension” refers to any method where two nucleic acid sequences become linked (e.g., hybridized) by an overlap of complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

### (ix) Proximity Ligation

A “proximity ligation” is a method of ligating two (or more) nucleic acid sequences that are in proximity with each other through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference).

A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (x) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

### (xi) PCR Amplification

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques well known in the art, such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

(xii) Label, Detectable Label, and Optical Label

The terms “detectable label” and “label” are used interchangeably herein to refer to a directly or indirectly detectable moiety that is associated with (e.g., conjugated to) a molecule to be detected, e.g., a probe for in situ assay, or analyte. The detectable label can be directly detectable by itself (e.g., radioisotope labels or optical labels such as fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature. For example, detectably labeled features can include a fluorescent, a colorimetric, or a chemiluminescent label attached to an analyte, probe, or bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore.

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLE

The following examples are included for illustrative purposes only and is not intended to limit the scope of the present disclosure.

### Example 1

### “Antidote” Method: Masking and Thereby Removing Highly Expressed Genes from the Combinatorial Read-Out Scheme in an Optically Crowded Tissue Sample and Detecting These Highly Expressed Genes in a Separate Round

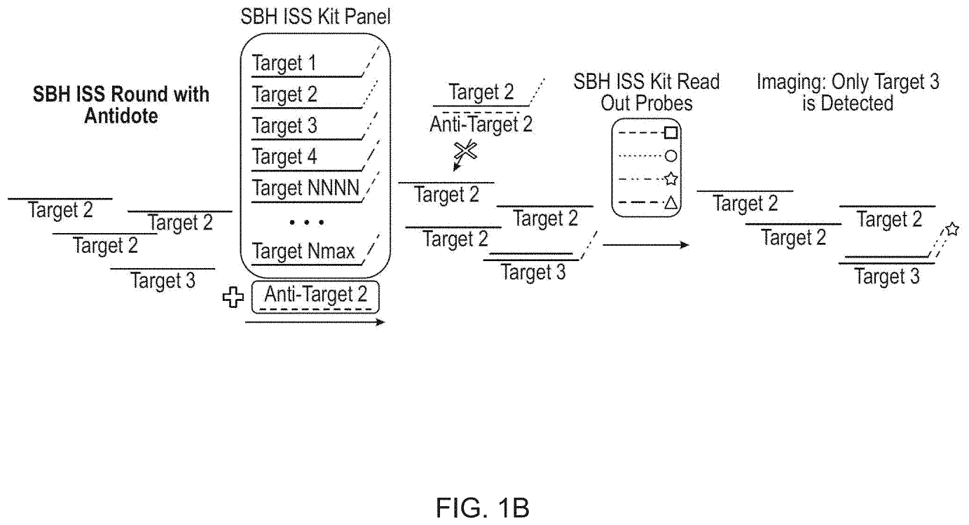

A tissue section was analyzed with a highly multiplexed padlock probe panel targeting multiple genes for detection using sequencing-by-hybridization (SBH). The padlock probes were circularized and amplified by RCA. During the decoding of the barcode sequences present in the RCA products, problems occurred in certain regions where too many signals were generated, which made optically resolving individual RCA products impossible and hence inhibited the combinatorial read out through the different cycles of the SBH decoding scheme (FIG. 2). FIG. 2 shows two fluorescence microscopy images of a colon tissue section undergoing in situ sequencing-by-hybridization, wherein padlock probes complementary to the target nucleic acid sequences have been amplified using RCA reactions. In the left image, all RCPs have been stained, and in the right image, all nuclei in the sample have been stained. The boxed region in the left image shows an area of very high expression, where decoding the signals in order to identify the genes which are expressed is difficult due to the signal crowding.

From visual inspection of some SBH cycles, it was observed that only 3 or 4 genes were responsible for causing this signal crowding. This was apparent because the cells which were especially full of RCA products appeared to contain RCA products with the same barcode sequence. It was not possible to optically resolve all of the individual RCA products within these cells, but it was possible to detect the signal codes of the bulk of RCA products in each cycle by simply visually following the cycles. The signal code sequences for these RCA products could thus be inferred (FIGS. 3A-3B).

FIGS. 3A-3B show fluorescence microscopy images from the decoding cycles of the ISS SBH reaction. In the circled areas, genes were overexpressed to such an extent that visual decoding is possible, wherein the decoding was based on the overall color of a cell in a specific cycle. In this situation, the signals are too strong to allow every single RCP within each cell to be observed throughout each of the cycles. Moreover, it is impossible to detect signals relating to the expression of other genes underneath those highly crowded signals, and thus information would be lost if the signal crowding issue were not addressed. In FIG. 3A, images from four decoding cycles are shown. In FIG. 3B, images from three decoding cycles are shown. The signal code sequences for the cells at A, B, C and D are shown, where R denotes a red signal, G denotes a green signal, W denotes a white signal, Y denotes a yellow signal, and “?” denotes a signal which cannot be identified. For example, the signal code sequence for the cells at A is RRGG (red-red-green-green) from the four decoding cycles of fluorescence hybridization of detectable probes. The genes corresponding to the detected signal code sequences are also shown, and antidote probes can then be prepared for these highly expressed selected genes. FIG. 3 thus provides an exemplary method of identifying target nucleic acid sequences for designing corresponding interfering agents (“antidote” probes).

This visual decoding resulted in the identification of 3 genes that were very highly expressed and caused signal crowding the sample. A further gene was also suspected to be highly expressed. Targets A and B in FIG. 3 were selected for preparation of corresponding antidote probes, and the two additional genes for selection of antidote probes were similarly identified, These 4 genes which had been identified as responsible for significant signal crowding were therefore selected, and antidote probes were prepared which were complementary to the hybridization probes corresponding to these 4 genes.

### Methods

### Multiplexed Padlock Probe and RCA In Situ, Inside Fixed Colon Tissue Sections

Fresh frozen colon tissue samples were cryo-sectioned at 10 μm and collected on ThermoFisher Superfrost glass slides. Slides were left to thaw at room temperature (RT) for 5 min. A fixation step was conducted by incubating slides in 3.7% PFA in 1× DEPC-PBS at RT for 45 min. The slides were then washed in 1× DEPC-PBS for 1 min to ensure PFA removal, before permeabilization in 0.1M HCl in DEPC H₂O with added pepsin of the final concentration 0.1 mg/ml for 90 sec at RT. Following this, the slides were then washed twice in 1× DEPC-PBS before dehydrating with ethanol series in 70% and 100% ethanol for 2 min each respectively. The slides were subsequently air dried at RT for 5 min before applying a Secure Seal chamber (Grace Bio-Labs) to each section. The sections were then rehydrated with 1× DEPC-PBS-T. To perform sample preparation, the Cartana Neurokit was used (Cartana, Sweden). For reverse transcription, a reaction mix with enzymes were mixed together and added to each tissue section in a secure seal chamber mounted on top of the tissue slides. Samples were incubated overnight at 37° C. RM1 was then removed from the secure seal chambers and a post fixation solution containing 3.7% PFA in lx DEPC-PBS was added to the samples and incubated at room temperature for 30 min. After post fixation, the samples were washed twice in 1× DEPC-PBS-T. For probe ligation, a reaction mix with enzymes and 50 nM of the Padlock probe were mixed and added into each secure seal chambers and incubated at 37° C. for 30 min followed by at 45° C. for 60 min. RM2 was then removed and samples were washed twice with 1× DEPC-PBS-T. For probe amplification, a reaction mix with polymerase were mixed and added to each tissue section in a secure seal. Samples were incubated overnight at 30° C. and then washed twice in 1× DEPC-PBS-T, after which the samples were ready for the in situ sequencing-by-hybridization reaction.

### In Situ Sequencing (ISS) by Hybridization of RCPs in Prepared Tissue Sections

For the first decoding cycle, 100 μl of a SBH mix containing 2×SSC, 20% Formamide and SBH-oligonucleotide G (CACA TGCGTCTATGTAGTGGAGCC TT AGAGAGTAGTACTTCCGACT, SEQ ID NO: 1), SBH-oligonucleotide A (CACA TGCGTCTATATAGTGGAGCC TT GTA GTA CAG CAG CAG CAT TGA GG, SEQ ID NO: 2), SBH-oligonucleotide T (CACA TGCGTCTATTTAGTGGAGCC TT CAA TCT AGT ATC AGT GGC GCA, SEQ ID NO: 3), SBH-oligonucleotide C (CACA TGCGTCTATCTAGTGGAGCC TT GGG CCT TAT TCC GGT GCT AT, SEQ ID NO: 4) and SBH-detection oligonucleotides Cy3-AGTCGGAAGTACTACTCTCT (SEQ ID NO: 5), Cy5-CCTCAATGCTGCTGCTGTACTAC (SEQ ID NO: 6), AF488-TGCGCCACTGATACTAGATTG (SEQ ID NO: 7), and TexR-ATAGCACCGGAATAAGGCCC (SEQ ID NO: 8) was used. The SBH-oligonucleotides represent hybridization probes according to the present disclosure and invention. The detection oligonucleotides represent reporter probes as defined herein. The sequencing reaction (e.g., to detect hybridized probes) was incubated for 60 min at 37° C. The sequencing mix was then removed and the tissue sections were washed in PBS-T 0.05% twice. Subsequently, the tissue sections were mounted with mounting medium and a cover slip and imaged using 20× objective Nikon microscope (Eclipse Ti2).

### Antidote Blocking of Some Genes During In Situ Sequencing (ISS) by Hybridization of RCPs in Prepared Tissue Sections and Subsequent Individual Barcode Detection

Antidote blocking was performed using gene specific antidote oligonucleotides of final concentration 1 uM in 100 ul of SBH mix containing 2× SSC, 20% Formamide and SBH-oligonucleotide G (CACA TGCGTCTATGTAGTGGAGCC TT AGAGAGTAGTACTTCCGACT, SEQ ID NO: 1), SBH-oligonucleotide A (CACA TGCGTCTATATAGTGGAGCC TT GTA GTA CAG CAG CAG CAT TGA GG, SEQ ID NO: 2), SBH-oligonucleotide T (CACA TGCGTCTATTTAGTGGAGCC TT CAA TCT AGT ATC AGT GGC GCA, SEQ ID NO: 3), SBH-oligonucleotide C (CACA TGCGTCTATCTAGTGGAGCC TT GGG CCT TAT TCC GGT GCT AT, SEQ ID NO: 4). The sequencing reaction was incubated for 60 min at 37° C. The sequencing mix was then removed and the tissue sections were washed twice in PBS-T. Sections were then incubated for 60 min at 37° C. with 100 ul of detection mix containing 2×SSC, 20% Formamide and SBH-detection oligonucleotides Cy3-AGTCGGAAGTACTACTCTCT (SEQ ID NO: 5), Cy5-CCTCAATGCTGCTGCTGTACTAC (SEQ ID NO: 6), AF488-TGCGCCACTGATACTAGATTG (SEQ ID NO: 7), and TexR-ATAGCACCGGAATAAGGCCC (SEQ ID NO: 8). The sequencing mix was then removed, and the tissue sections were washed twice in PBS-T 0.05%. The tissue sections were mounted with mounting medium and a cover slip and imaged using 20× objective Nikon microscope (Eclipse Ti2). Detection of the genes for which antidote probes had been provided was performed in a separate SBH cycle using 100 ul of SBH mix containing 2× SSC, 20% Formamide and gene specific oligonucleotides of final concentration 0.1 uM instead of SBH-oligonucleotides.

### Results

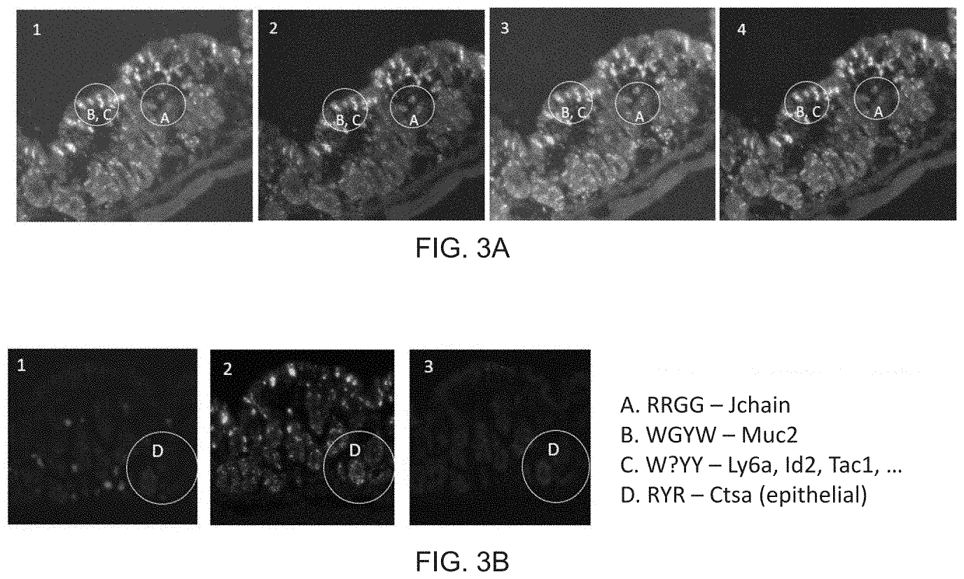

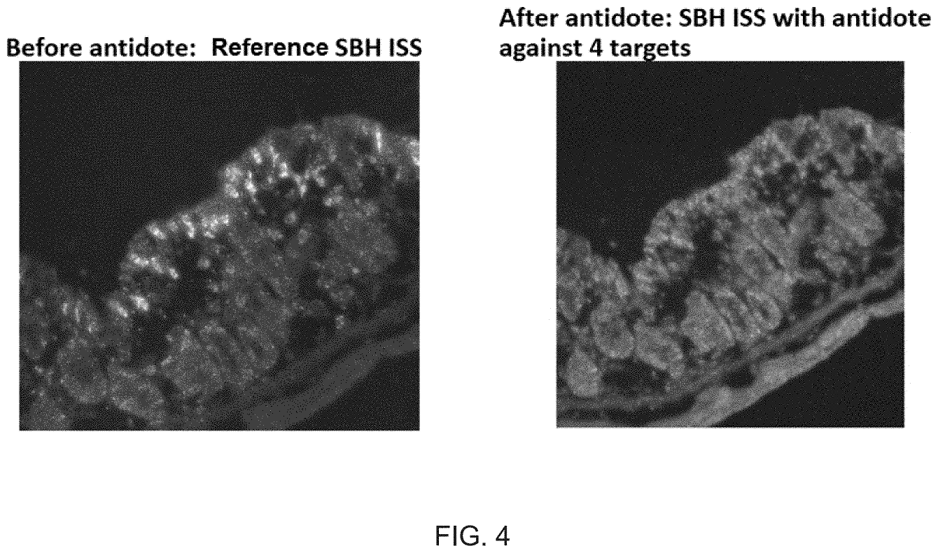

The antidote probes complementary to the 4 identified hybridization probes were added to the plurality of hybridization probes present in the SBH kit. An elevated concentration of antidote probes was used, relative to the concentration of the corresponding hybridization probes, to ensure that all of the hybridization probes were bound by complementary antidote probes, such that they could not hybridize to the barcode sequences in the RCA products. This lead to the masking of the RCA products generated from these high expressed genes. The samples were imaged and it was observed that the strong signals from the high expressed genes were no longer present, and that a lot of underlying signals that were previously not visible before were now visible (FIG. 4 and FIG. 5).

FIG. 4 shows fluorescence microscopy images before (left) and after (right) the use of antidote probes against 4 highly expressed selected genes. The antidote probes were applied to every cycle of the SBH kit reaction mix, and all of the RCA products from the other, non-selected genes that were now better visible after masking the high expressed gene RCPs were sequenced. It can be seen that the antidote probes successfully prevented signals from the selected genes from being generated, and therefore reduced signal crowding. These lower expressed genes that were optically masked by the high expressed genes could now be decoded, which was not possible without the antidote probes.

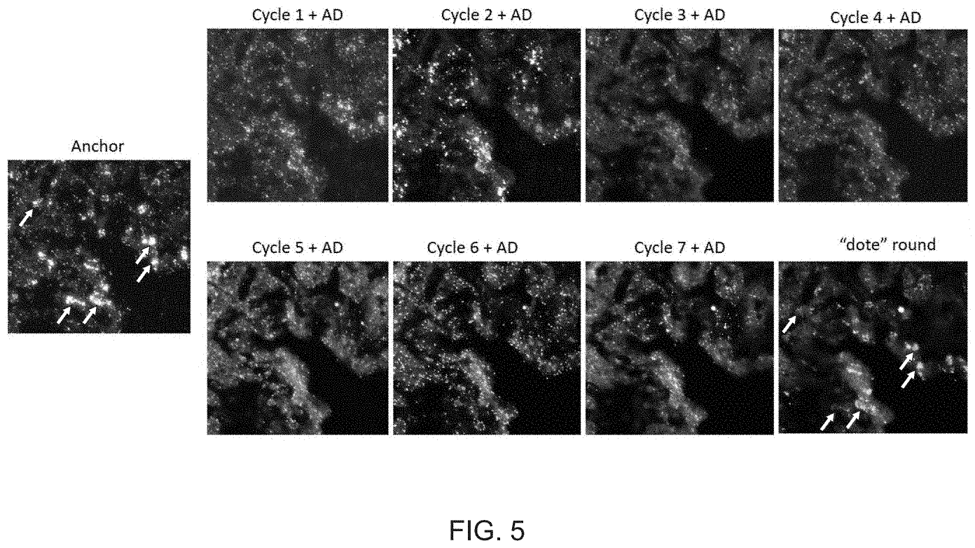

When all of the necessary SBH cycles were completed (7 cycles were used in total) and the lower expressed genes were decoded, 4 separate hybridization probes for the barcode sequences corresponding to the 4 selected genes were hybridized to the sample, in order to visualize only these 4 genes individually in a separate decoding round, referred to as a “dote round” (FIG. 5 and FIG. 6).

FIG. 5 shows fluorescence microscopy images from all 7 decoding cycles of the ISS SBH reaction with the antidote probes (AD), followed by a separate “dote” round to detect only the genes for which signal generation was prevented by the use of antidote probes. In the anchor image, all RCPs were stained. Arrows indicate the high expressed genes for which antidote probes were prepared. Signals from these genes are not generated during the decoding cycles due to the antidote probes. Instead, these genes are detected separately in the “dote” round using additional hybridization probes.

FIG. 6 shows fluorescence microscopy images from the “dote” round. The channels have been separated so that the genes can be visualized individually, 1 per channel. This “dote” round occurs after all of the decoding cycles of SBH ISS reaction (where the antidote probes are present) are completed. The genes which were masked are detected using 4 separate hybridization probes. This data can then be overlaid with the other ISS data. After quantifying the signals from the highly expressed genes in the “dote” round, the data could be overlaid so as to visualize both very low and very high expressed genes in the same tissue section.

1. A method for nucleic acid sequence detection, comprising:
(a) in any suitable order, contacting (i) sample comprising a first target nucleic acid sequence and a second target nucleic acid sequence, (ii) a first probe capable of hybridizing to the first target nucleic acid sequence, (iii) a second probe capable of hybridizing to the second target nucleic acid sequence, and (iv) an interfering agent, wherein:
the first and second target nucleic acid sequences are different, and
hybridization of the first probe to the first target nucleic acid sequence is not interfered by the interfering agent, whereas hybridization of the second probe to the second target nucleic acid sequence is interfered by the interfering agent; and
(b) detecting a signal indicative of the hybridization of the first probe to the first target nucleic acid sequence in the sample, whereas a signal indicative of the hybridization of the second probe to the second target nucleic acid sequence in the sample is not detected or is detected at a lower level compared to a reference signal detected without the interfering agent interfering with hybridization,
thereby detecting the first target nucleic acid sequence in the sample.

2. The method of claim 1, wherein:
the sample comprises a plurality of first target nucleic acid sequences that are different from each other, and
the contacting step comprises contacting the sample with a plurality of first probes each capable of hybridizing to one of the plurality of first target nucleic acid sequences.

3.-4. (canceled)

5. The method of claim 1, wherein the first and second probes are contacted with the interfering agent to form a second probe/interfering agent hybridization complex, before the sample is contacted with the first and second probes and the interfering agent.

6.-11. (canceled)

12. The method of claim 1, wherein the interfering agent comprises a sequence complementary to a sequence of the second probe, or a sequence complementary to a sequence of the second target nucleic acid sequence.

13. The method of claim 1, wherein the interfering agent hybridizes to the second probe but not to the first probe.

14.-15. (canceled)

16. The method of claim 1, wherein the interfering agent hybridizes to the second target nucleic acid sequence but not to the first target nucleic acid sequence.

17.-20. (canceled)

21. The method of claim 1, wherein the first probe and/or the second probe directly or indirectly bind to a detectably labelled detection probe.

22. The method of claim 1, wherein the first probe and/or the second probe comprise one or more overhangs that do not hybridize to the first and second target nucleic acid sequences, respectively, and wherein at least one of the one or more overhangs is capable of hybridizing to a detectably labelled detection probe.

23.-25. (canceled)

26. The method of claim 1, wherein the first and second target nucleic acid sequences correspond to a first analyte and a second analyte, respectively, in the sample, and wherein the first analyte is less abundant than the second analyte in the sample.

27.-35. (canceled)

36. The method of claim 1, further comprising:
removing the first probe hybridized to the first target nucleic acid sequence in the sample; and
contacting the sample with the second probe but not the interfering agent, and detecting a signal indicative of the hybridization of the second probe to the second target nucleic acid sequence in the sample, thereby detecting the second target nucleic acid sequence in the sample.

37. (canceled)

38. The method of claim 1, further comprising prior to the contacting step, contacting the sample with the first and second probes but not with the interfering agent, wherein a signal indicative of the hybridization of the first probe to the first target nucleic acid sequence in the sample spatially overlaps with and/or has a lower amplitude than the reference signal which is indicative of the hybridization of the second probe to the second target nucleic acid sequence in the sample without the interfering agent.

39.-40. (canceled)

41. The method of claim 1, wherein the first and/or second target nucleic acid sequences are comprised in a product of a nucleic acid analyte in the sample, or a product of a labelling agent or a polynucleotide probe that directly or indirectly binds to an analyte in the sample.

42. (canceled)

43. The method of claim 41 wherein the product is a rolling circle amplification product.

44. (canceled)

45. The method of claim 1, wherein the first and/or second target nucleic acid sequences are comprised in an RCA product of a circular or padlock probe that hybridizes to a DNA or RNA analyte in the sample.

46.-50. (canceled)

51. The method of claim 1, wherein the first and/or second target nucleic acid sequences are detected in situ in the sample.

52.-53. (canceled)

54. The method of claim 1,
wherein a unique signal code sequence is assigned to each target nucleic acid sequence,
wherein a set of probes is provided for decoding of each signal code sequence for each target nucleic acid sequence,
wherein each probe in a set comprises the same recognition sequence that hybridizes to the target nucleic acid sequence and a detection hybridization region or the absence of a detection hybridization region,
wherein the detection hybridization region or the absence thereof may be the same or different among probes in the set, wherein the detection hybridization region, if present, is specific for a detection probe comprising a detectable label or lacking a detectable label, and
wherein the probes of the set are used sequentially in multiple cycles of decoding in a pre-determined sequence which corresponds to the signal code sequence.

55. The method of claim 54, wherein a given cycle of decoding comprises contacting the sample with a probe library comprising a probe of each set of probes, wherein the probe of each set corresponds to the given cycle of decoding.

56. The method of claim 55, wherein the method comprises contacting the sample with an interfering agent or a set of interfering agents in multiple cycles of decoding, wherein the same interfering agent or set of interfering agents is used with the probe library in each cycle of decoding.

57. The method of claim 56, wherein the interfering agent or an interfering agent of the set of interfering agents interferes with hybridization of different second probes to the same corresponding second target nucleic acid sequences in different cycles of decoding.

58. The method of claim 57, wherein the different second probes share a binding sequence that hybridizes to the same second target nucleic acid sequence but comprise different binding sequences for different detectably labelled detection oligonucleotides.

59.-91. (canceled)
